(19) United States
(12) Patent Application Publication
(10) Pub. No.: US 2024/0166602 A1
(43) Pub. Date: May 23, 2024

(54) SUBSTITUTED DIARYL COMPOUND AS WELL AS PREPARATION METHOD AND APPLICATION THEREOF

(71) Applicant: SHANDONG FIRST MEDICAL UNIV & SHANDONG ACADEMY OF MEDICAL SCIENCES, Jinan City (CN)

(72) Inventors: Bo LIU, Jinan City (CN); Qingqiang YAO, Jinan City (CN); Haijiao CHEN, Jinan City (CN); Xinmei YANG, Jinan City (CN); Ying ZHI, Jinan City (CN); Ying LI, Jinan City (CN); Haiyang WANG, Jinan City (CN); Zhengguo CUI, Jinan City (CN); Xiaoxiang LIU, Jinan City (CN); Guodong HU, Jinan City (CN); Tiandi DING, Jinan City (CN); Feipeng ZHANG, Jinan City (CN)

(21) Appl. No.: 18/540,293

(22) Filed: Dec. 14, 2023

Related U.S. Application Data
(63) Continuation of application No. PCT/CN2022/082764, filed on Mar. 24, 2022.

(30) Foreign Application Priority Data
Jun. 15, 2021 (CN) 2021106587654

(51) Int. Cl. C07D 211/14; A61P 35/00; C07C 217/64; C07D 207/06; C07D 211/82

(52) U.S. Cl. CPC C07D 211/14; A61P 35/00; C07C 217/64; C07D 207/06; C07D 211/82

(57) ABSTRACT

The invention relates to the field of medicinal chemistry, in particular to a substituted diaryl compound shown as a formula (I), a preparation method of the substituted diaryl compound, a medicinal preparation containing the substituted diaryl compound and medical application of the substituted diaryl compound. Pharmacological test results show that the substituted diaryl compound disclosed by the invention has a good inhibition effect on cells of human lung cancer (A549), human ovarian cancer (SKOV3), human melanoma (A375) and human colon cancer (LOVO). The formula (I) is shown in the specification:

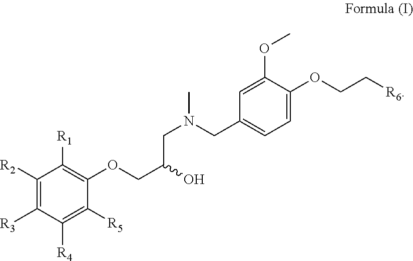

### TECHNICAL FIELD

The present invention relates to the field of medicinal chemistry, specifically to a type of substituted diaryl compounds, their preparation methods, their pharmaceutical preparations and their medicinal uses.

### BACKGROUND

1-(2-ethoxyphenoxy)-3-(3-methoxy-4-(2-(4-methylpiperidin-1-yl)ethoxy)benxyl)(methyl)amino)propan-2-ol, CAS No: 2125657-10-3, molecular formula C₂₈H₄₂N₂O₅, structural formula:

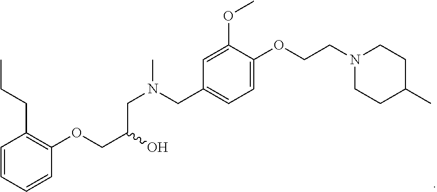 [SMILES: CCCc1ccccc1OCC(O)CN(C)Cc1ccc(OCCN2CCC(C)CC2)c(OC)c1]

The currently published literature only contains the physical and chemical properties of this compound, and there is no disclosure of its pharmacological effects, let alone any literature reports on its anti-tumor effects.

### SUMMARY

The technical problem solved by the present invention is to provide a type of substituted diaryl compounds and pharmaceutically acceptable salts thereof, their optical isomers, their preparation methods, pharmaceutical compositions and their application in the preparation of drugs for treating cancer.

In order to solve the technical problems of the present invention, the present invention provides the following technical solutions. The first aspect of the technical solution of the present invention is to provide a compound of general formula (I) or a pharmaceutically acceptable salt thereof, or an optical isomer thereof:

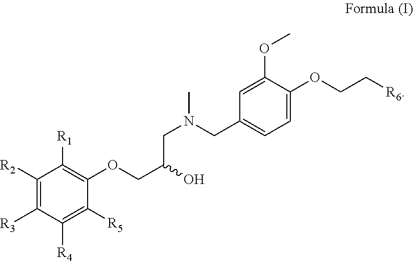

Wherein, R₁ represents —OC₂H₅, —H, —CH(CH₃)₂, —Br, —CF₃, —OCH₃, —F, Cl, —CH₃
R₂ represents —F, —CF₃, —Br, —NHCOCH₃, —Cl, —H, —OCH₃, —CH(CH₃)₂
R₃ represents —H, —CH₃, —Cl, —Br, —NHCOCH₃, —C₃H₇, —F, —C₁₄H₂₀, —OCH₃
R₄ represents —H, Br, —CF₃, —Cl
R₅ represents —H, —Cl, —I, -—Br,
R₆ represents

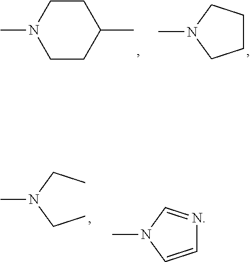

Preferably, the above-mentioned compound or its pharmaceutically acceptable salt or its optical isom, The described R₁ represents —H, —CH(CH₃)₂, —Br, —Cl

R₂ represents —F, —Br, —Cl, —H, —CH(CH₃)₂, —CF₃
R₃ represents —H, —CH₃, —Cl, —Br, —C₁₄H₂₉
R₄ represents —H, —Br, —CF₃, —Cl
R₅ represents —H, —Cl, —I, -—Br,
R₆ represents

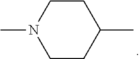

More preferably, the above-mentioned compound or its pharmaceutically acceptable salt or its optical isomer, The described R₁ represents —H; R₂ represents —CF₃; R₃ represents —Br; R₄ represents —H; R₅ represents —H; R₆ represents

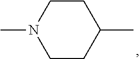

Or R₁ represents —H; R₂ represents —H; R₃ represents —C₁₄H₂₉; R₄ represents —H;
R₅ represents —H; R₆ represents

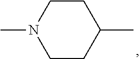

Or R₁ represents —H; R₂ represents —H; R₃ represents —Cl; R₄ represents —Br; R₅ represents —H;
R₆ represents

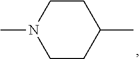

Or R₁ represents —Br; R₂ represents —H; R₃ represents —Br; R₄ represents —H; R₅ represents —Br; R₆ represents

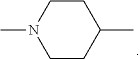

More preferably, the above-mentioned compound or its pharmaceutically acceptable salt or its optical isom. The described R₁ represents —H; R₂ represents —CF₃; R₃ represents —Br; R₄ represents —H; R₅ represents —H; R₆ represents

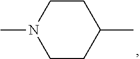

Or R₁ represents —H; R₂ represents —H; R₃ represents —C₁₄H₂₉; R₄ represents —H;
R₅ represents —H; R₆ represents

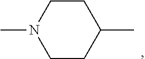

Or R₁ represents —Br; R₂ represents —H; R₃ represents —Br; R₄ represents —H; R₅ represents —Br;
R₆ represents

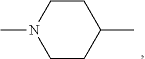

More preferably, the above-mentioned compound or its pharmaceutically acceptable salt or its optical isomer. The described R₁ represents —H; R₂ represents —CF₃; R₃ represents —Br; R₄ represents —H; R₅ represents —H; R₆ represents

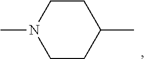

Or R₁ represents —Br; R₂ represents —H; R₃ represents —Br; R₄ represents —H; R ₅ represents —Br;
R₆ represents

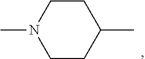

Pharmaceutically acceptable salts of any of the above compounds of the present invention includes salt of organic acid, salt of inorganic acid, salt of organic alkali or salt of inorganic alkali. The organic acid includes acetic acid, trifluoroacetic acid, methylsulfonic acid, toluenesulfonic acid, maleic acid, succinic acid, tartaric acid, citric acid, fumaric acid; the inorganic acid includes hydrochloric acid, hydrobromic acid, nitric acid, sulfuric acid, phosphoric acid; the organic alkali includes meglumine, glucosamine; the inorganic alkali includes the alkaline compounds of sodium, potassium, barium, calcium, magnesium, zinc, and lithium.

Any of the above compounds in the invention is racemes or its optical isomer is its levoisomer or dextroisomer.

The second aspect of the technical solution of the present invention is to provide a preparation method for the compound described in the first aspect.

The compound of general formula of the present invention can be prepared by the following method:

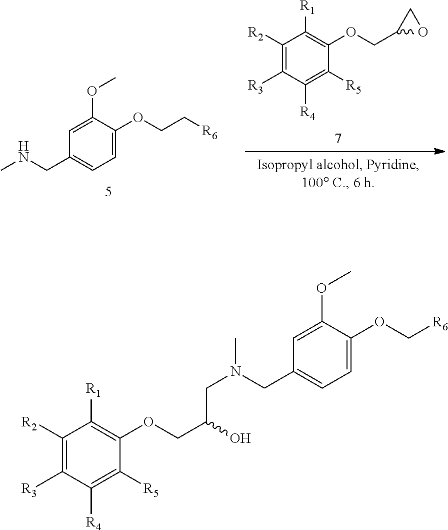

Wherein, R₁ represents —OC₂H₅, —H, —CH(CH₃)₂, —Br, —CF₃, —OCH₃, —F, —Cl —CH₃

R₂ represents —F, —CG₃—Br, —NHCOCH₃, —Cl, —H, —OCH₃, —CH(CH₃)₂
R₃ represents —H, —CH₃, —Cl, —Br, —NHCOCH₃, —C₃H₇, —F, —C₁₄H₂₉, —OCH₃
R₄ represents —H, —Br, —CF₃, —Cl
R₅ represents —H, —Cl, —I, —BR,
R₆ represents

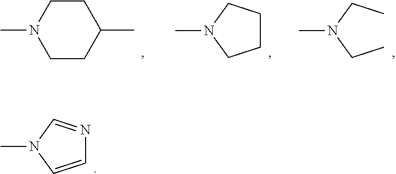

Synthesis of the series of compounds of general formula (I): dissolve substituted phenoxymethyloxirane (7) (1.0 mmol) and compound (5) (1.2 mmol) in isopropanol (15 mL) under nitrogen protection, add a catalytic amount of pyridine, heat to reflux for 6 hours, and detect the disappearance of the raw material by TLC. The reaction solution was diluted with ethyl acetate, and the organic phase was washed with water and saturated brine in sequence, dried over anhydrous sodium sulfate, filtered, and the solvent was evaporated under reduced pressure. The crude product was separated and purified by silica gel column chromatography (mobile phase: dichloromethane-methanol=20:1) to obtain target compound (I) with a yield of 80%-90%.

The fourth aspect of the technical solution of the present invention provides the application of the compounds described in the first aspect and the pharmaceutical composition described in the third aspect to the preparation of a drug for treating cancer. The described cancer is lung cancer or ovarian cancer or melanoma or colon cancer.

The clinical method of administration of compound de scribed in the invention can be oral administration, injection., etc. The clinical dosage of the compound of the present invention is 0.01-1000 mg/day, and may deviate from this range depending on the severity of the disease or the dosage fort

Beneficial technical etiects: The present invention provides a series of compounds with novel structures and anti-cancer effects, which compounds are effective against lung cancer, ovarian cancer, melanoma or colon cancer.

### DETAILED DESCRIPTION OF EMBODIMENTS

The following specific embodiments will further illustrate the present invention so that those skilled in the art can better understand the present invention, but do not limit the present invention in this way.

Synthesis of the target compound in the embodiment of present invention: Dissolve substituted phenoxymethyloxirane (7) (1.0 mmol) and compound. (5) (1.2 mmol) in isopropanol (15 mL) under nitrogen protection , add a catalytic amount of pyridine, heat to reflux for 6 hours, and detect the disappearance of the raw material by TIC. The reaction solution was diluted with ethyl acetate, and the organic phase was washed with water and saturated brine in sequence, dried over anhydrous sodium sulfate, filtered, and the solvent was evaporated under reduced pressure. The crude product was separated and purified by silica gel column chromatography (mobile phase: dichloromethane-methanol=20:1) to obtain target compound.

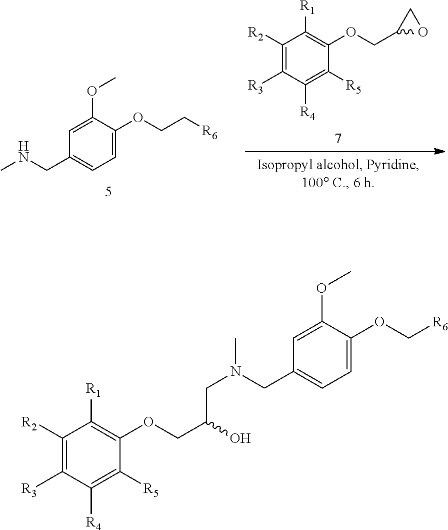

Compound (5) is synthesized through 4 steps of reaction using vanilline (1) as the raw material.

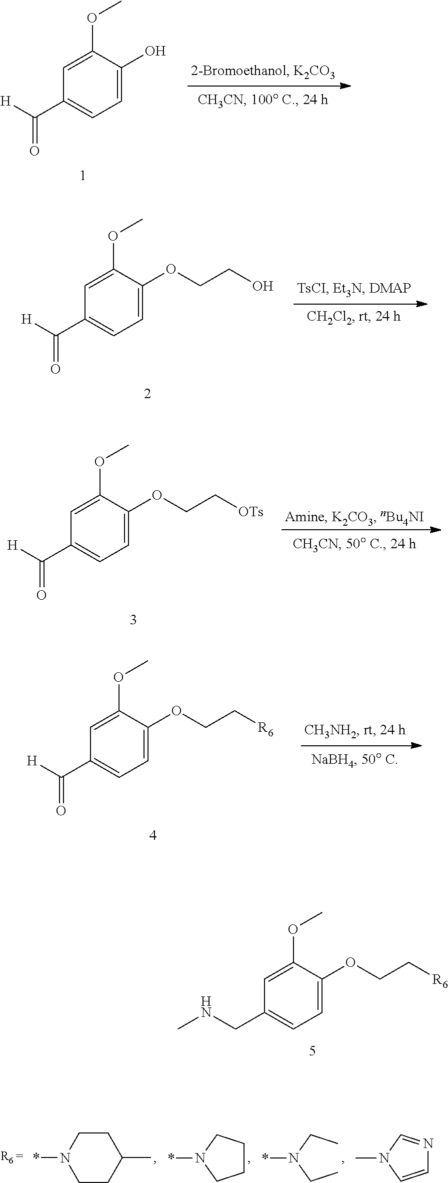

The 4 types of compound (5) obtained is shown in the following table;

TABLE 1
Detailed Structure of Compound (5)

| No | Structure |
|---|---|
| 5-1 | 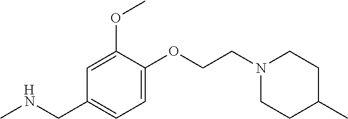 |
| 5-2 | 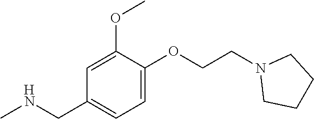 |
| 5-3 | 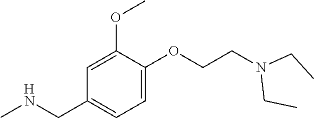 |
| 5-4 | 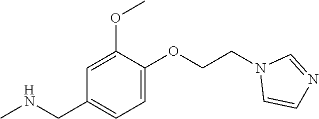 |

Substituted phenoxyl methyloxirane (7) is synthesized from the nucleophilic substitution between the phenol of different substitutions on the benzene ring and epibromohydrin. The compound series obtained is shown in the following table:

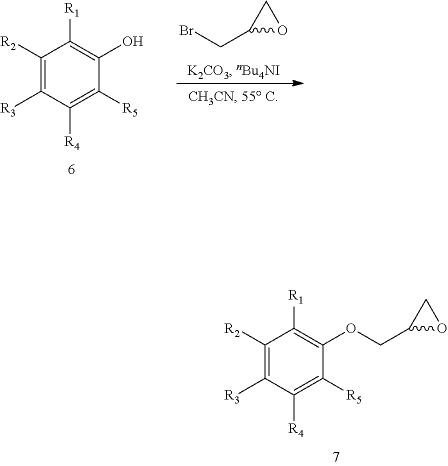

TABLE 2
Detailed Structure of Compound (7)

| No | Structure |
|---|---|
| 7-1 | 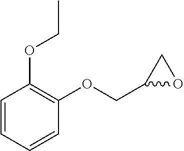 |
| 7-2 | 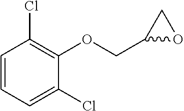 [SMILES: Clc1cccc(Cl)c1OCC1CO1] |
| 7-3 | 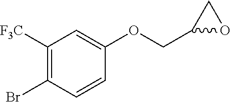 [SMILES: FC(F)(F)c1cc(OCC2CO2)ccc1Br] |
| 7-4 | 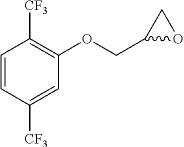 [SMILES: FC(F)(F)c1ccc(C(F)(F)F)c(OCC2CO2)c1] |
| 7-5 | 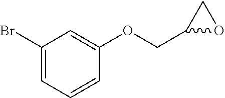 [SMILES: Brc1cccc(OCC2CO2)c1] |
| 7-6 | 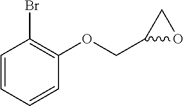 [SMILES: Brc1ccccc1OCC1CO1] |
| 7-7 | 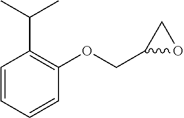 |
| 7-8 | 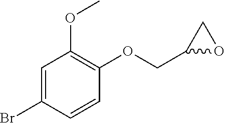 |
| 7-9 | 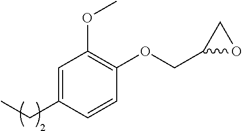 |
| 7-10 | 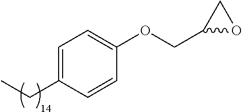 |
| 7-11 | 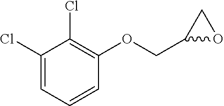 [SMILES: Clc1cccc(OCC2CO2)c1Cl] |
| 7-12 | 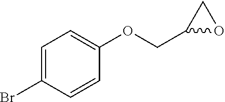 [SMILES: Brc1ccc(OCC2CO2)cc1] |
| 7-13 | 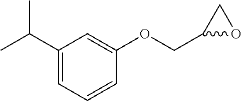 |
| 7-14 | 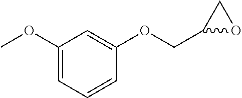 |
| 7-15 | 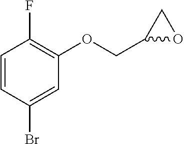 [SMILES: Fc1ccc(Br)cc1OCC1CO1] |
| 7-16 | 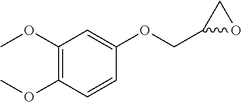 |
| 7-17 | 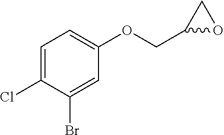 [SMILES: Clc1ccc(OCC2CO2)cc1Br] |
| 7-18 | 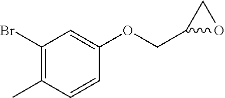 |
| 7-19 | 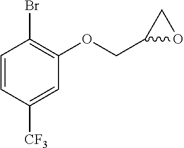 [SMILES: FC(F)(F)c1ccc(Br)c(OCC2CO2)c1] |
| 7-20 | 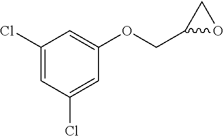 [SMILES: Clc1cc(Cl)cc(OCC2CO2)c1] |
| 7-21 | 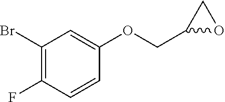 [SMILES: Fc1ccc(OCC2CO2)cc1Br] |
| 7-22 | 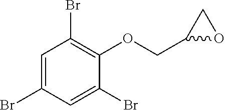 [SMILES: Brc1cc(Br)c(OCC2CO2)c(Br)c1] |
| 7-23 | 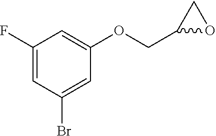 [SMILES: Fc1cc(Br)cc(OCC2CO2)c1] |
| 7-24 | 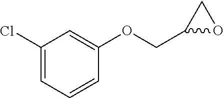 [SMILES: Clc1cccc(OCC2CO2)c1] |
| 7-25 | 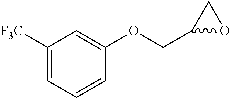 [SMILES: FC(F)(F)c1cccc(OCC2CO2)c1] |
| 7-26 | 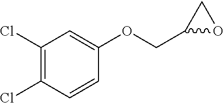 [SMILES: Clc1ccc(OCC2CO2)cc1Cl] |
| 7-27 | 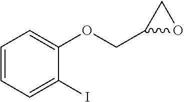 [SMILES: Ic1ccccc1OCC1CO1] |
| 7-28 | 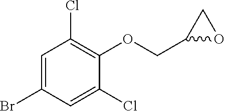 [SMILES: Clc1cc(Br)cc(Cl)c1OCC1CO1] |
| 7-29 | 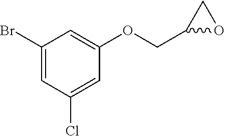 [SMILES: Clc1cc(Br)cc(OCC2CO2)c1] |
| 7-30 | 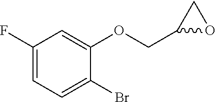 [SMILES: Fc1ccc(Br)c(OCC2CO2)c1] |
| 7-31 | 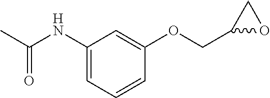 |
| 7-32 | 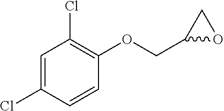 [SMILES: Clc1ccc(OCC2CO2)c(Cl)c1] |
| 7-33 | 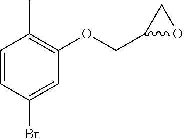 |
| 7-34 | 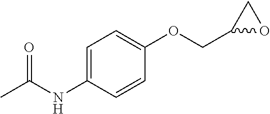 |
| 7-35 | 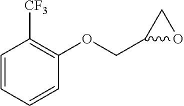 [SMILES: FC(F)(F)c1ccccc1OCC1CO1] |
| 7-36 | 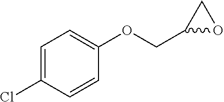 [SMILES: Clc1ccc(OCC2CO2)cc1] |
| 7-37 | 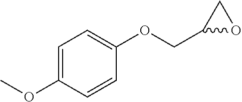 |
| 7-38 | 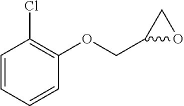 [SMILES: Clc1ccccc1OCC1CO1] |
| 7-39 | 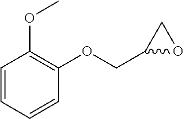 |

### EMBODIMENT 1

synthesis of 1-(2-ethoxyphenoxy)-3-((3-methoxy-44 -(4-tnethylpiperidin-1yl)ethoxy)benzyl)(methyl)amino)propan-2ol (SAMS10)

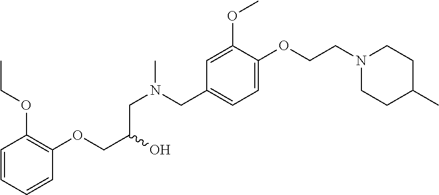 [SMILES: CCOc1ccccc1OCC(O)CN(C)Cc1ccc(OCCN2CCC(C)CC2)c(OC)c1]

Compound 5 used is 1-(3-methoxy-4-(2-(4-methylpiperidin-1-yl)ethoxy)phenyl)-N-methylmethanamine (5-1.1).

Compound 7 used is 2(2-methoxyphenoxy)methyl)oxirane(7-1).

Dissolve 2-((2-ethoxylphenoxyl) methyl) oxirane (7-1) (194 mg, 1.0 mmol) and 1-(3-methoxyl-4-(2-(4-methyl piperidine-1-base) ethoxyl) phenyl)-N-methyl methylamine (5-1) (354 mg, 1.2 mmol) in isopropanol (15 mL), and, with the protection of nitrogen, add pyridine (8.0μL, 0.1 mmol.) of catalyst dosage. Apply heating reflux for 6 h and the TLC test (developer: dichloramethane-methanol=10:1) raw material disappears. Dilute the reaction liquid with ethyl acetate, and wash the organic phase with. water and saturated saline solution in order. Dry it with anhydrous sodium sulfate and filter it. The solvent was evaporated under reduced pressure. The crude product was separated and purified by silica gel column chromatography (mobile phase: dichloromethane-methanol=20:1), a colorless oily substance (437.9 mg 90%) was obtained. ¹H NMR (CDCl₃, 600 MHz,) δ (ppm): 6.89 (m,7H), 4.09 (m, 7H), 3.84 (s, 3H), 3.59 (d, J=19.80 Hz, 1H), 3.47 (d, J=19.80 Hz, 1H), 3.05 (d, J=17.40 Hz, 2H), 2.90 (t, J=9.60 Hz, 2H), 2.61 (m, 2H), 2.28 (s, 3H), 2.19 (m, 2H), 1.66 (d, J=21.0 Hz, 2H), 1.38 (m, 6H), 0.94 (d, J=8.40 Hz, 3H). ¹³C NMR (CDCl₃, 150 MHz) δ (ppm): 149.49, 149.39, 148.72, 147.33, 131.62, 122.12, 121.27, 121.10, 115.80, 113.84, 113.16, 112.48, 72.91, 66.61 (2C), 64.51, 62.45, 59.60, 57.25, 55.94, 54.37 (2C), 42.43, 33.84 (2C), 30.39, 21.76, 14.93, IR (KBr, cm⁻¹): 2924, 2871, 2851, 2794, 2360, 2340, 1592, 1512, 1494, 1460, 1419, 1368, 1321, 1272, 1217, 1138, 1035, 980, 863, 803, 772, 670. HRMS (t SI):. m/z calcd. for C₂₈H₄₃N₂O₅ (M×H)^{×}: 487.3172. found: 487.3199.

The preparation method of compounds CHJ02029-CHJ05004 in Examples 2-58 is the same as that in Example 1. The difference is that different compounds 5 and 7 are used for synthesis. Specifically, the raw materials of compound 5 and compound 7. used in each embodiment are as follows in the corresponding embodiment record.

### EMBODIMENT 2

synthesis of 1-(2,6-dichlorophenoxy)-3-((3-methoxy-4-(2-(4-methylpiperidin-1-yl)ethoxy)benzyl) (methyl)amino)propan-2-ol (CHJ02029)

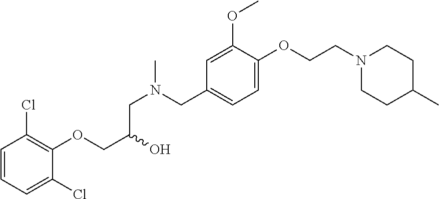 [SMILES: COc1cc(CN(C)CC(O)COc2c(Cl)cccc2Cl)ccc1OCCN1CCC(C)CC1]

Compound 5 used is 1-(3-methoxy-4-(2-(4-methylpiperldin-1-yl)ethoxy)phenyl)-N-methylmethanamine (5-1).

Compound 7 used is 2((2,6-dichlorophenoxy)methyl)oxirane (7-2).

Colorless oil, yield 88%, ¹H NMR (CD₃OD, 400 MHz)δ (ppm):7.36 (d,J=8.0 Hz, 2H), 7.08 (t,j=8.0 Hz, 1H), 7.01 (s, 1H), 6.87 (m, 2H), 4.15 (m, 3H), 3.99 (m, 2H), 3.80 (s, 3H), 3.59 (m, 2H), 3.14 (d, J=11.20 Hz, 2H), 2.92 (t, J=5.60 Hz, 2H), 2.74 (m, 1H), 2.59 (dd, J=12.80, 7.60Hz, 1H), 2.32. (m, 5H), 1.70 (d, J=12.80 Hz, 2H), 1.45 (s, 1H), 1.30 (m 2H), 0.95 (d, J=6.4 Hz, 3H). ¹³CNMR (CD₃OD,100 MHz)δ (ppm) 151.27, 149.68, 147.30, 131.70, 129.04, 128.89 (3C), 125.31, 121.62, 113.84, 113.09, 75.76, 67,86, 66.25, 62,02, 59.27, 56.86, 55.00, 53.81 (2C), 41.86, 33.04 (2C), 29.92, 20.60. IR (KBr, cm⁻¹): 2947, 2926, 2872, 2841, 2792, 2360, 2340, 1651, 1592,1511, 1475, 1455, 1367, 1286, 1262, 1230, 1127, 1036, 979, 937, 863, 807, 670. HRMS (ESI): m/z calcd for C₂₆H₃₇Cl₂N₂O₄ (M+H)⁺: 511.2130. found: 511.2047.

### EMBODIMENT 3

synthesis .of 1-(4-bromo-3-(trifluoromethyl)phenoxy)-3-((3-methoxy-4-(2-(4-methylpiperidin-1-yl)ethoxy)benzyl)(methyl)amino)propan-2-ol (CHJ02049)

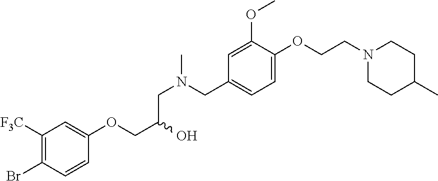 [SMILES: COc1cc(CN(C)CC(O)COc2ccc(Br)c(C(F)(F)F)c2)ccc1OCCN1CCC(C)CC1]

Compound 5 used is 1-(3-methoxy-4-(2-(4-methylpiperdin-1-yl)ethoxy)phenyl)-N-methylmethanamine (5-1).

Compound 7 used is 2-((4bromo-3-(trifluoromethyl)phenoxy)methyl)oxirane (7-3).

Colorless oil, yield 87%, ¹H NMR (CD₂OD, 400 MHz)δ (ppm):7.64 (d, J=8.80 Hz, 1H), 7,26 (s, 1H), 7.02 (d, J=8.80 Hz, 1H), 6.95 (s, 1H), 6.83 (q, J=8.0 Hz, 2H), 4.08 (m, 4H), 3.91 (m, 1H), 3.75 (s, 3 H), 3.50 (q,J=12.80 Hz, 2H), 3.03 (d,J=11.60 Hz, 2H), 2.80 (t, J=5.60 Hz, 2H), 2.62 (dd, J=12.40, 5.60 Hz, 1H), 2.48 (dd, J=12.40, 6.40 Hz, 1H), 2.32 (s, 3H), 2.17 (t, 11.60 Hz, 2H), 1.66 (d, J=12.40 Hz, 2H), 1.39 (s, 1H), 1.29 (m, 2H), 0.94 (d, J=6.40 Hz, 3H). ¹³CNMR (CD₃OD,100 MHz)δ (ppm):158.33, 149,62, 147.45, 135.7 (2C), 131,91, 121,46 (2C), 118.91, 114.35. 114.29, 113.48, 113.06, 70.99, 67.29, 66.69, 62.29, 58.70, 57.12, 54.94, 54.00 (2C), 42.25, 33.48 (2C), 33.25, 20.75. IR (KBr, cm⁻¹): 2926, 2872, 2849, 2793, 2370, 2323, 1684, 1651, 1556, 1512, 1474, 1455, 1419, 1367, 1330, 1313, 1260, 1235, 1139, 1035, 980, 936, 879, 809, 753. HRMS (ESI): m/z calcd for C₂₇H₃₇BrF₃N₂O₄ (M+H)⁺: 589.1889. found: 589.2404.

### EMBODIMENT 4

synthesis of 1-(2,5-bis(trifluoromethyl)phenoxy)-3-((3-methoxy-4-(2-(4-methylpiperidin-1-yl)ethoxy)benzyl)(methyl)amino)propan-2-ol (CHJ02050)

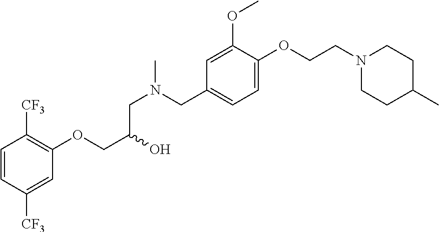 [SMILES: COc1cc(CN(C)CC(O)COc2cc(C(F)(F)F)ccc2C(F)(F)F)ccc1OCCN1CCC(C)CC1]

Compound 5 used is 1-(3-methoxy-4-(2-(4-methylpiperidin-1-yl)ethoxy)phenyl)-N-methylmethanamine (5-1).

Compound 7 used is 2-((2,5-bis(trifluoromethyl)phenoxy)methyl)oxirane (7-4).

White s(olid, yield 85%, mp: 7072° C., ¹H NMR (CD₃OD, 400 MHz)δ (ppm): 7.77 (d, J=8.0 Hz, 1H), 7.46 (s, 1H), 7.38 (d, J=8.0 Hz, 6.96 (s, 1H), 6.83 (q, J8.0 Hz, 2H), 4.13 (m, 5H), 3.76 (s, 3H), 3.51 (m, 2H), 3.03 (d, J=11.20 Hz, 2H), 2.80 (t, J5.60 Hz, 2H), 2.63 (m, 2H), 2.30 (s, 3H) 2.17 (t, J=11.60 Hz, 2H), 1.66 (d, J=12.40 Hz, 2H), 1.40 (s, 1H), 1.29 (m, 2H), 0.94 (d, J=6.40 Hz. 3H), ¹³CNMR (CD3OD, 100 MHz) δ (ppm): 157.29, 149.66, 147.41, 131.89, 127.72, 127.67, 121.67, 121.40. 116.72, 116.68, 113.59, 112.97, 109.94, 109.90, 71.45, 67.17, 66.71, 62.28, 59.06, 57.11, 54.90, 53.98 (2C), 41,93, 33.46 (2C), 30.24, 20.73, IR (KBr, cm⁻¹): 3562, 3354, 2945, 2877, 2831, 2800, 1624, 1595, 1517, 1463, 1435, 1330, 1259, 1232, 1174, 1132, 1087,1043, 1022, 962, 910, 866, 833, 804, 750, 673.HRMS. (EST): calcd for C₂₈H₃₇F₆N₂O₄ (M+4)⁺: 579.2658. found: 579.2549.

### EMBODIMENT 5

synthesis of 1-(3-bromophenoxy)-3-((3-methoxy-4-(2-(4-methylpiperidin-1-yl)ethoxy)benzyl) (methyl)amino)propan-2-ol (CHJ03001)

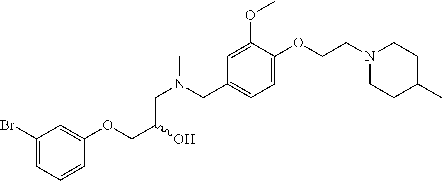 [SMILES: COc1cc(CN(C)CC(O)COc2cccc(Br)c2)ccc1OCCN1CCC(C)CC1]

Compound 5 used is 1-(3-methoxy-4-(2-(4-methylpiperidin-1-yl)ethoxy)phenyl)-N-methylmethanamine (5-1).

Compound 7 used is 2-((3-bromophenoxy)methyl)oxirane (7-5).

Colorless oil, yield 86%,¹H NMR (CDCl₃, 400 MHz)δ (ppm): 7.10 (m,3H), 6.82 (m, 4H), 4.14 (m, 3H), 3.94 (d, J=4.80 Hz, 2H), 3.85 (s, 3H), 3.62 (d, J=13.60 Hz, 1H), 3.45 (d, J=13.20 Hz, 1H), 2.98 (d, J=11.20 Hz, 2H), 2.84 (t, J=6.0 Hz, 2H), 2.62 (m, 1H), 2.49 (dd, J=12.40, 3.60 Hz, 1H), 2.29 (s, 3H), 2.11 (t, J=11.60 Hz, 2H), 1.64 (d, J=12.40 Hz, 2H), 1.30 (m, 3H), 0.93 (d, J=4.0 Hz, 3H). ¹³CNMR (CDCl₃, 100 MHz) δ (ppm): 159.54, 149.44, 147.60, 131.18, 130.53, 124.07, 122.76, 121.30, 117.86, 113.58, 112.97, 112.49, 70.63, 66.81, 66.04, 62.37, 59.25 57.40, 55.97, 54.49 (2C), 42.26, 34.19 (2C), 30.57, 21.87. IR (KBr, cm⁻¹): 2947, 2924, 2871, 2846, 2792, 2360, 2340, 11651, 1591, 1572, 1512, 1476, 1463, 1459, 1368, 1324, 1283, 1261, 1229, 1157, 1138, 1090, 1035, 991, 936, 861, 804, 800, 674. HRMS (ESI); calcd for C₂₆H₃₈BrN₂O₄ (M+H)⁺: 521.2015. found: 521.1945.

### EMBODIMENT 6

synthesis of 1-(2-bromophenoxy)-3-((3-methoxy-4-(2-(4-methylpiperidin-1-yl)ethoxy)benzyl) (methyl)amino)propan-2-ol (CHJ03003)

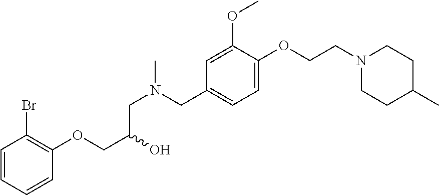 [SMILES: COc1cc(CN(C)CC(O)COc2ccccc2Br)ccc1OCCN1CCC(C)CC1]

Compound 5 used is 1-(3-methoxy-4-(2-(4-methylpiperidin -1yl)ethon)phenyl)-N-methylmethanamine (5-1).

Compound 7 used is 2-((2-bromophenox methyl)oxirane (7-6).

Colorless oil,yield 90%, ¹H NMR (CDCl³, 400 MHz)δ (ppm): 7.52 (d,J=7.60 Hz,1H), 7.23 (dJ=8.0 Hz,1H), 6.85 (m, 5H), 4.14 (t,J=6.0 Hz, 3H.), 4.03 (d,J=4.40 Hz, 2H), 3.84 (s, 3H), 3.72 (q,J=7.20 Hz, 1H), 3.63 (d,J=12.80 Hz, 1H), 3.47 (d, J=12.80 Hz, 1H), 2,98 (d,J=11.20 Hz, 2H), 2.84 (t, J=6.40 Hz, 2H), 2.72 (m, 1H), 2.59 (dd, J=12.0, 3.60 Hz, 1H), 2.31 (s, 3H), 2.11 (t, J=11.20 Hz, 2H), 1.64 (d, J=12.40 Hz, 2H), 1.27 (m, 3H), 0.93 (d, J=6.0 Hz, 3H). ¹³CNMR (CDCl₃, 100 MHz)δ (ppm): 155.10, 149.41, 147.54, 133.30, 131.32, 128.46, 122.19, 121.33, 113.54, 112,94, 112.52, 112.41, 71.39, 66.74, 66.25, 62.47, 59.35, 58.43, 57.39, 55.96, 54.47, 42.43, 34.17, 30.57, 21.86, 18.45. IR (KBr, em⁻¹):2947, 2924, 2871, 2844, 2792, 2361, 2340, 1589, 1513 1480, 1462, 1417, 1368, 1323, 1276, 1261, 1232, 1158, 1138, 1084, 1053, 1030, 979, 939, 872, 806, 749. HRMS (ESI): m/zcalcdfir C₂₆H₃₈BrN₂O₄ (M+H)⁺: 521.2015. found: 521,1943.

### EMBODIMENT 7

synthesis of 1(2-isopropylphenoxy)-3-((3-methoxy-4-(2-(4-methylpiperidin-1yl)ethoxy)benzyl) (methyl)amino)propan-2-ol (CHJ03004)

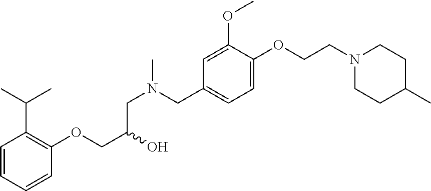 [SMILES: COc1cc(CN(C)CC(O)COc2ccccc2C(C)C)ccc1OCCN1CCC(C)CC1]

Compound 5 used is 1-(3-methoxy-4-(2-(4-methylpiperidin -1-yl)ethoxy)phenyl)-N-methylmethanamine (5-1).

Compound 7 used is 2-((2-isopropylphenoxy)methyl)oxirane (7-7).

Colorless oil,yield 87%, ¹H NMR (CDCl³, 400 MHz)δ (ppm): 7.2 (d,J=7.60 Hz,1H), 7.13 (t,J=7.60 Hz,1H), 6.92 (t,J=7.20 Hz, 1H.), 6.82 (m, 4H, 4.14(m,3H), 3.00 (m, 2H), 3.94 (s, 3H), 3.65 (d,J=13.20 Hz, 1H), 3.45 (d,J=12.80 Hz 1H), 3.26 (m, 1H), 2.98 (d, J=11.20 Hz, 2H), 2.84 (t, J=6.40 Hz, 2H), 2.69 (m, 1H), 2.53 (dd, J=12.0, 3.20 Hz, 1H), 2.31 (s, 3H), 2.11 (t,J=11.2 Hz, 2H), 1.64 (d,J=12.0 Hz, 2H), 1.30 (m, 3H), 1.19 (d, J=6.80 Hz, 6H), 0.96 (d,J=6.0 Hz, 3H). ¹³CNMR (CDCl₃, 100 MHz)δ (ppm): 155,82, 149.43, 147.56, 137.05, 131.31, 126.55, 126.07, 121.26, 120.89, 112.93, 112.45, 111.30, 70.34, 66.78, 66.35, 62.47, 59.67, 57.40, 55.94, 54.48, (2C), 42.36, 34.19 (2C), , 30.57, 26.90, 22.64 (2C), 21.88.IR (KBr, cm⁻¹): 2950, 2925, 2870, 2792, 2360, 2340, 1597, 1513, 1491, 1452, 1418, 1365, 1323, 1261, 1238, 1193, 1138, 1088, 1033, 1030, 980, 937, 878, 822, 805, 751. HRMS (ESI): m/z calcdfor C₂₉H₄₅N₂O₄ (M+H)⁺: 485.3379. found: 485.3330.

### EMBODIMENT 8

synthesis of 1-(4-bromo-2-methoxypbenoxy)-3-((3-methoxy-4-(2-(4-methylpiperidin-1-yl)ethoxy) benzyl)(methyl)amino)propan-2-ol (CHJ03005)

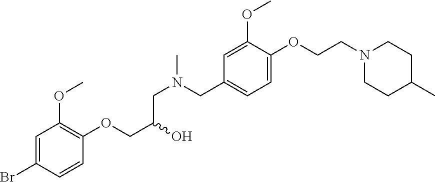 [SMILES: COc1cc(CN(C)CC(O)COc2ccc(Br)cc2OC)ccc1OCCN1CCC(C)CC1]

Compound 5 used is 1-(3-Methoxy-4-(2-(4-methylpiperidin-1-yl)ethoxy)phenyl)-N-methylmethanamine(5-1).

Compound. 7 used is 2-((4-bromo-2-methoxyphenoxy)methyl)oxirane (7-8).

Colorless oil,yield 90%, 1H NMR (CD₃OD, 400 MHz)δ (ppm): 7.06 (s, 1H), 7.00 (d,J=8.40 Hz,1H), 6.95(s,1H), 6.83 (m, 3H), 4.10 (m, 3H), 3.96 (dd, J=9.60, 5.60 Hz, 1H), 3.85 (dd, J=9.60, 5.60 Hz, 1H), 3.77 (d, J=12.0 Hz, 6H), 3.53 (m, 2H), 3.06 (d, J=11.60 Hz, 2H), 2.83 (t, J=5.88 Hz, 2H), 2.63 (dd, J=13.44. 5.60 Hz, 1H), 2.49 (dd, J=13.44, 6.80 Hz, 1H), 2.31 (s, 3H), 2.20 (t, J=11.60 Hz, 2H), 1.67 (d, J=12.80 Hz, 2H), 1.40 (s, 1H), 1.28 (m, 2H), 0.94 (d,J=6,40 Hz, 3H). ¹³C NMR (CD₃OD, 100 MHz)δ (ppm): 150.42, 149.62, 147.91, 147.36, 131.88, 123.29, 121.47, 115.25, 114.88, 113.54, 113.05,112.79, 71.91, 67.50, 66.61, 62.22, 58.89, 57.07, 55.34, 54.94, 53.96 (2C). 42.13, 33.39 (2C), 30.18, 20.71, IR (KBr, cm⁻¹): 2946, 2924, 2843, 2792, 2360, 2340, 1589, 1556,1539, 1506, 1459, 1418, 1398, 1364, 1324, 1255, 1225, 1183, 1136, 1084, 1029, 936, 857, 797, 670. HRMS (ESI): m/z calcdfor C₂₇H₄₀BrN₂O₅ (M+H)⁺: 551.2101. found: 551.2094.

### EMBODIMENT 9

synthesis of 1-((3-methoxy-4-(2-(4-methylpiperidin-1-yl)ethoxy)benzyl)(methyl)amino)-3-(2-methoxy-4-propylphenoxy)propan-2-ol (CHJ03011)

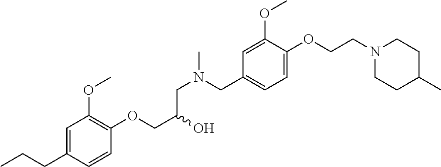 [SMILES: CCCc1ccc(OCC(O)CN(C)Cc2ccc(OCCN3CCC(C)CC3)c(OC)c2)c(OC)c1]

Compound 5 used is 1-(3-methoxy-4-(2-(4-methylpiperidin-1-yl)ethoxy)phenyl)-N-methylmethanamine (5-1),

Compound 7 used is 2-((2-methoxy-4-propylphenoxy)mathyl)oxirane (7-9).

White solid,yield 83%,mp: 45-47° C., ¹H NMR (CD₃OD, 400 MHz) δ (ppm): 6.97 (s, 1H), 6.87 (d, J=8.0 Hz,1H), 6.80 (m,3H), 6.69 (d, J=8.0 Hz, 1H), 4.10 (m, 3H), 3.96 (dd, J=9.60, 3.60 Hz, 1H), 3.84 (dd, J=9.60, 6.40 Hz, 1H), 3.78 (d, J=13.60 Hz, 6H), 3.54 (m, 2H), 3.05 (d, J=11.60 Hz, 2H), 2.82 (t, J=5.60 Hz, 2H), 2.61 (dd, J=12.80, 5.20 Hz, 1H), 2.51 (q, J=7.20 Hz, 3H), 2.30 (s, 3H), 2.19 (t, J=11.60 Hz, 2H), 1.63 (m, 4H), 1.40 (s, 1H), 1.28 (m, 2H), 0.94 (d, J=7.20 Hz, 6H). ¹³C NMR (CD₃OD, 100 MHz)δ (ppm): 1.49.66, 149,33, 147.40, 146.45, 136.1.0, 131.85. 121.50, 120.41, 113.95, 113.60, 113.07, 112,54, 72.26, 67.62, 66.67, 62.16, 59.08, 57.08, 55.14, 54.96, 53.97 (2C), 42.01, 37.26, 33.41 (2C), 30.19, 24.48, 20.71, 12.68. IR (1(KBr, cm⁻¹): 2922. 2868, 2791, 2360, 2340, 1597, 1516, 1458, 1419, 1371, 1330, 1261, 1230, 1138, 1091, 1031, 970, 850, 804, 750, 646, 553, 489, HRMS (EST); m/zcalcdfor C₃₀H₄₇N_{2d}O₅ (M+H)⁺: 515.3485. found: 515.3423.

### EMBODIMENT 10

synthesis of 1((3-methoxy-4-(2-(4-methylpiperidin-1- yl)ethoxy)benzyl)(methyl)amino -3-(4-pentadecylphenoxy)propan-2-ol (CHJ03012)

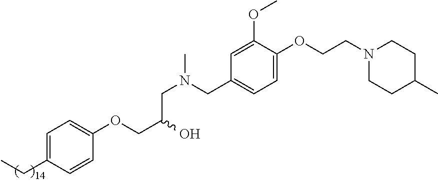

Compound 5 used is 1-(3-methoxy-4(2-(4-methylpiperidin-1-yl)ethoxy)phenyl)-N-methylmethanamine (5-1).

Compound 7 used is 2-((4-pentadecylphenoxy)methyl)oxirane (7-10).

White solid,yield 85%,mp: 40-42° C., ¹H NMR (CD₃OD, 400 MHz)δ (ppm): 7.13 (t, K=7.60 Hz, 1H), 6.97 (s, 1H), 6.84 (m, 2H), 6.72 (m, 3H), 4.11 (m, 3H), 3.98 (m, 1H), 3.86 (m, 1H), 3.76 (s, 3H), 3.55 (m, 2H), 3.05 (d,J=11.60 Hz, 2H), 2.81((t, J=5.60 Hz, 2H), 2.59 (m, 4), 2.30 (m, 3H), 2.18 (t, J=11.60 Hz, 2H), 1.64 (m, 4H), 1.28 (s, 27H), 0.94 (d, J=6.40 Hz, 3H), 0.89 (t, J=6.0 Hz, 3H), ¹³C NMR (CD₃OD, 100 MHz)δ (ppm): 159.02, 149.69, 147.42, 144.23, 131.93, 128.79, 121.47, 120.62, 114.35, 113.65, 113.08, 111.38, 70.34, 67.53, 66.71, 62.22, 59.19, 57.11, 54.98, 53.98 (2C), 53.38, 42.07, 35.56, 33.43 (2C), 31.67, 31.21, 30.21, 29.35 (6C), 29.20, 29.07, 28.91, 22.33, 20.72, 13.04. (KBr, cm⁻¹): 2924, 2852, 2794, 2360, 2340, 1591, 1514, 1458, 1367, 1325, 1263, 1151, 1085, 1035, 937, 869, 806, 775, 694. HRMS (ESI): m/z calcdfor C₄₁H₆₉N₂O₄ (M+H)⁺: 653.5257, found: 653.5163.

### EMBODIMENT 11

synthesis of 1-(2,3-dichlorophenoxy)-3-((3-methoxy-4-(2-(4-methylpiperidin-1-yl)ethoxy)benzyl)(methyl)amino)propan-2-ol (CHJ03013)

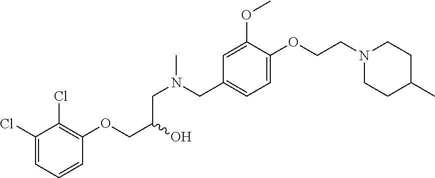 [SMILES: COc1cc(CN(C)CC(O)COc2cccc(Cl)c2Cl)ccc1OCCN1CCC(C)CC1]

Compound 5 used is 1-(3-methoxy-4-(2-(4-methylpiperidin-1-yl)ethoxy)phenyl)-N-methylmethanamine (5-1).

Compound 7 used is 2-((2,3-diellorophenoxy)methyl)oxirane (7-11).

Colorless oil,yield 88% ¹H NMR (CD₃OD, 400 MHz) δ (ppm): 7.20 (t,J=8.11 Hz,1H), 7.08 (t,J=8.08 Hz,1H) 6.96 (m,2H), 6.82 (q,J=8.05 Hz, 2H), 4.05 (m, 5H), 3.75 (s, 3H), 3.52 (s, 2H), 3.03 (d,J=11.43 Hz, 2H), 2.80 (t,J=5.65 Hz, 2H), 2.69 (dd, J×12.82, 5.4 Hz, 1H), 2.56 (dd,J=12.73, 7.02 Hz, 1H), 2.32 (s, 3H), 2.17 (t,J=11.7 Hz, 2H), 1.65 (d,J=12.71 Hz, 2H), 1.38 (s, 1H), 1.27 (m, 2H), 0.93 (d,J=6.28 Hz, 3H). ¹³CNMR (CD₃O D, 100 MHz) δ (ppm): 155.92, 149.65, 147.41, 133.15, 131.90, 127.52, 121.91, 121.45, 121.32, 113.56, 113.03, 111,43, 71.63, 67.36, 66,65, 62,35, 58.90, 57.11, 54,97, 53.97 (2C), 42.20, 33.46 (2C), 30.2.2, 20.76. IR (KBr, cm⁻¹): 2947, 2926. 2872, 2841, 2792, 2360, 2340, 1651, 1592, 1511, 1475, 1455, 1367, 1286, 1262, 1230, 1127, 1036, 979, 937, 863, 807, 670. HRMS (ESI): calcd for C(₂₆H₃₇Cl₂N₂O₄ (M+H)⁺: 511.2130. found: 511.2095.

### EMBODIMENT 12

synthesis of 1-(4-bromophenoxy)-3-((3-methoxy-4-(2-)4-methylpiperidin-1-yl)ethoxy)benzyl) (methyl)amino)propan-2-ol (CHJ03014)

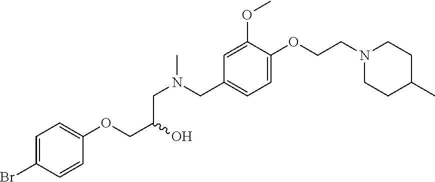 [SMILES: COc1cc(CN(C)CC(O)COc2ccc(Br)cc2)ccc1OCCN1CCC(C)CC1]

Compound 5 used is 1-(3-methoxy-4-(2-(4-methylpiperidin-1-yl)ethoxy)phenyl)-N-methylmethanamine (5-1),

Compound 7 used is 2-((4-hromophenoxy)methyl)oxirane (7-12).

Colorless oil,yield 81%, ¹H NMR (CD₃OD, 400 MHz) δ (ppm): 7.35 (d,J=8.0 Hz, 2H), 6.95 (s, 1H), 6.82 (m, 4H), 4.10 (t,J=5.60 Hz, 2H), 4.05 (m, 1H), 3.95 (dd,J=9.60. 3.20 Hz, 1H), 3.84 (dd,J=9.60, 6.0 Hz, 1H), 3.77 (s, 3H), 3.50 (q,J=12.80 Hz, 2H), 3.03 (d,J=11.20 Hz, 2H), 2.80 (t,J=5.60 Hz, 2H), 2.61 (dd,J=12.83. 6.0 Hz,1H), 2.48 (dd,J=12.80, 7.20 Hz, 1H), 2.30 (s, 3H), 2.17 (t,J=11.60 Hz, 2H), 1.65 (d,J=12.80 Hz, 2H), 1.40 (s, 1H), 1.28 (m, 2H), 0.93 (d,J=6.40 Hz, 3H). ¹³CNMR (CD₃OD, 100 MHz) δ ppm): 158.28, 149.64, 147.44, 131.89 (2C), 131.89, 121.49, 116.19 (2C), 113.56. 113.10, 112.35, 70.66, 67.42, 66.70, 62.27, 58.96, 57,12, 54.88, 53.99 (2C). 42.21. 33.47 (2C), 30.23. 20.76. IR (KBr, cm⁻¹): 2947. 2925, 2871, 2844, 2792, 2360, 2331, 1591, 1556, 1512, 1489, 1458, 1418, 1368, 1322, 1285, 1245, 1157, 1074, 1034, 980, 937, 879, 863, 821, 756, 647. HRMS (ES!): calcdfor C₂₆H₃₈BrN₂O₄ (M+H)⁺: 521.2015. found: 521.1975.

### EMBODIMENT 13

synthesis of 1-(3-isopropylphenoxy)-3-((3-methoxy-4-(2-(4-methylpiperidin-1-yl)ethoxy)benzyl) (methyl)amino)propan-2-ol (CHJ03015)

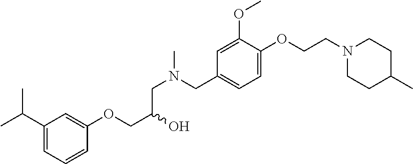 [SMILES: COc1cc(CN(C)CC(O)COc2cccc(C(C)C)c2)ccc1OCCN1CCC(C)CC1]

Compound. 5 used is 1-(3-methoxy-4-(2-(4-methylpiperidin-1-yl)ethoxy)phenyl)-N-methylmethanamine (5-1).

Compound 7 used is 2-((3-isopropylphenoxy)methyl)oxirane (7-13).

Colorless oil,yield 89%, ¹H NMR (CD₃OD, 400 MHz) δ (ppm): 7.15 (t,J =7.60 Hz, 1H), 6.97 (s, 1H), 6.82 (m, 4H), 6.69 (d,J=8.0 Hz, 1H), 4.10 (m, 3H), 3.97 (m, 1H), 3.86 (m, 1H), 3.76 (s, 3H), 3.54 (m,:2H), 3.03 (d,J=11.20 Hz, 2H), 2.82 (m, 3H), 2.63 (dd,J=12.80, 5.60 Hz, 1H), 2.51 (dd,J=12.40, 6.8 Hz, 1H), 2.30 (s, 3H), 2.17 (t,J=11.60 Hz, 2H), 1.65 (d,J=12.40 Hz, 2H), 1.40 (s, 1H), 1.24 (m, 8H), 0.94 (d,J=6.0 Hz, ³H). ¹³CNMR(CD₃OD, 100 MHz) δ (ppm): 159.09, 150.37, 149.68, 147.44, 131.90, 128.90, 121.47, 118.55, 113.62, 113.09, 112.54, 111.36, 70.39, 67.54, 66.74, 62.21, 59.25, 57.12, 54.99 (3C), 42.05, 34.05, 33.46 (2C), 30.23, 23.03 (2C), 20.75, IR (KBr, cm⁻¹): 2952, 2925, 2871, 2844, 2792, 2360, 2340, 1606, 1588, 1513, 1486, 1460, 1418, 1366, 1320, 1262, 1233, 1286, 1138, 1088, 1037, 1003, 980, 940, 870, 804, 789, 754, 700. HRMS (ESI): - m/zealed for C₂₉H₄₅N₂O₄ (M+H)³⁰ : 485.3379. found: 485.3296.

### EMBODIMENT 14

synthesis of 1-((3-methoxy-4-(2-(4-methylpiperidin-1-yl)ethoxy)benzyl)(methyl)amino)-3-(3-methoxyphenoxy)propan2-ol (CHJ03017)

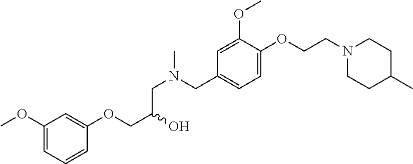

Compound 5 used is 1-(3methoxy-4-(2(4-methylpiperidin-1-yl)ethoxy)phenyl)-N-methylmethanamine (5-1).

Compound 7 used is 2-((3-methoxyphenoxy)methyl)oxirane (7-14).

Colorless oil,yield 83%, ¹H NMR (CD₃OD, 400 MHz) δ (ppm): 7.13 (t,J=8.0 Hz, 1H), 6.96 (s, 1H), 6.83 (m, 2H), 6.48 (m, 3H), 4.09 (m, 3H), 3.96 (dd,J=9.60, 6.80 Hz, 1H), 3.85 (dd,J=9.20, 6.0 Hz, 1H), 3.75 (d,J=7.60 Hz, 6H), 3.52. (m, 2H), 3.03 (d,J=11.60 Hz, 2H), 2.80 (t,J=5.60 Hz, 2H), 2.61(dd,J=12.80, 5.60 Hz, 1H), 2.49 (dd,J=12.80, 720 Hz, 2H), 2.30 (s. 3H), 2.16 (t,J=11.60 Hz, 2H), 1.65 (dd,J=12.40 Hz, 2H), 1.40 (s,1H.), 1.27 (m, 2H), 0.93 (d,J=6.40 Hz, 3H). ¹³CNMR(CD₃OD,100 MHz) δ (ppm): 160.97, 160.23, 149.65, 147.42, 131.,90, 129.48, 121.46, 113.58, 113.05, 106.35, 106.02, 100.65, 70.42, 67.48, 66.69, 62.23, 59.13, 57.12, 54.98, 54.28, 53.99 (2C). 42.10, 33.46 (2C), 30.23, 20.76.IR (KBr, cm⁻¹): 2947, 2925, 2872, 2837, 2792, 2360, 2340, 1593, 1559, 1513, 1492, 1455, 1418, 1368, 1334, 1287, 1264, 1231, 1201, 1154, 1083, 1036, 980, 940, 834, 807, 762, 687, HRMS (ESI): calcdfor C₂₇H₄₁N₂O₅ (M+J)⁺: 473.3015. found: 473.2947.

### EMBODIMENT :15

1-(5-bromo-2-fluorophenoxy)-3-(3-methoxy-4-(2-(4-methylpiperidin-1yl)ethoxy) benzyl)(methyl)amino)propan-2-ol (CHJ03018)

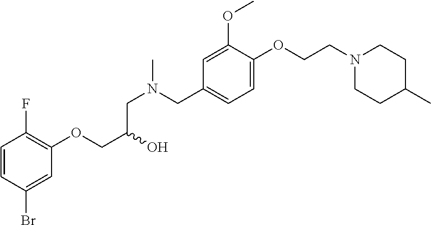 [SMILES: COc1cc(CN(C)CC(O)COc2cc(Br)ccc2F)ccc1OCCN1CCC(C)CC1]

Compound 5 used is 1-(3-methoxy-4-(2-(4-methylpiperidin-1-yl)ethoxy)phenyl)-N-methylmethanamine (5-1).

Compound 7 used is 2((5-bromo-2-fluorophenoxy)methyl)oxirane (7-15).

Colorless oil,yield 85%,¹H NMR (CD₃OD, 400 MHz) δ (ppm): 7.23 (d,J=7.20 Hz, 1H), 7.02 (m, 3H), 6.85 (dd,j=22.0, 8.0 Hz, 2H), 4.09 (m, 4H), 3.94 (dd,J=10.0, 5.60 Hz, 1H), 3.78 (s, 3H), 3.50 (m, 2H), 3.05 (d,J=11.60 Hz, 2H), 2.81 (t,J=5.60 Hz, 2H), 2.63 (dd,J=12.80, 5.60 Hz, 1H), 2.50 (dd, J=12,85, 6.51 Hz, 1H), 2,31 (s, 3H), 2.18 (t,J=12.0 Hz, 2H), 1.66 (d,J=13.20 Hz, 2H), 1.41 (s, 1H),1.28 (m, 2H), 0.94 (d,J=6.0 Hz, 3 H). ¹CNMR(CD₃OD),100 MHz) δ (ppm): 150.62, 149,66, 147.40, 131.94, 123.66, 123.59, 121.44, 118.06, 117.16, 116.96, 115.94, 113.59, 112.97, 72.04, 67.36, 66.68, 62.24, 58.85, 57.10, 54.98, 53.98 (2C), 42.10, 33.44 (.2C), 30.22, 20.72. IR (KBr, cm⁻¹): 2947, 2925, 2872, 2845, 2794, 2360, 2340, 1607, 1511, 1459, 1417, 1404, 1369, 1323, 1303, 1262, 1231, 1138, 1117, 1090, 1020, 962, 935, 877, 837, 803, 755, 627. HRMS (ESI): m/calcdfor C₂₆H₃₇BrFN₂O₄ (M+H)⁺: 539.1921. found: 539.1911.

### EMBODIMENT 16

synthesis of 1-(3,4-dimethoxyphenoxy)-3-((3-methoxy-4-(2-(4-methylpiperidin-1-yl)ethoxy)benzyl)(methyl)amino)propan-2-ol (CHJ03019)

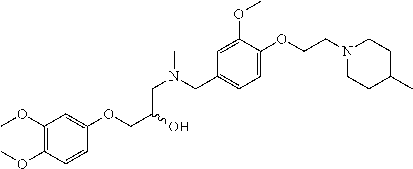

Compound 5 used is 1-(3-methoxy-4-(2-(4-methylpiperidin-1-yl)ethoxy)phenyl)-N-methylmethanamine (5-1).

Compound 7 used is 2((3,4-dimethoxyphenoxy)methyl)oxirane (7-16).

Colorless oil,yield 87%,¹H NMR (CD₃OD, 400 MHz) δ (ppm): 6.97 (s, 1H), 6.84 (m, 3H), 6.54 (s, 1H), 6.40 (d,J=8.55 Hz, 1H), 4.08 (m, 3H), 3,93 (dd,J=9.57, 3.33 Hz, 1H), 3.83 (m,1H), 3.77 (d,J=8.88 Hz, 9H), 3.52 (q,J=12.85 Hz, 2H), 3.05 (d,J=11.47 Hz, 2H), 2.82 (t, J=5.55 Hz, 2H), 2.62 (dd,J=12.84, 5.5 Hz, 1H, 1H), 2,49 (dd,J=12.77, 7.05 Hz, 1H), 2.31 (s, 3H), 2.19 (t,J=11.69 Hz, 2H), 1.66 (d,J=12.59 Hz, 2H), 1.40 (s, 1H), 1.27 (m, 2H), 0.94 (d, J=6,29 Hz, 3H). ¹³CNMR(CD₃OD,100 MHz) δ (ppm); 153.95, 150,09, 149.63, 147.39, 143.54, 131.88, 121.48, 113.57, 113.07, 112.86, 104.22, 100.91, 70.91, 67.53, 66.61, 62.22, 59.10, 57.08, 55.91, 54.97 (2C), 53.97 (20, 42.12, 33.39 (2C), 30.18, 20.72. IR (KBr, cm⁻¹): 2926, 2871, 2850, 2794, 2360, 2340, 1700, 1651, 1611, 1596, 1513, 1418, 1368, 1320, 1261, 1229, 1199, 1162, 1138, 1029, 981, 943, 875, 804, 764, HRMS (EST): m/zcalcdfor C₂₈H₄₃N₂O₆ (M+H)⁺: 503.3121. tbund: 501.2817.

### EMBODIMENT 17

synthesis of 1-(3-bromo-4-chlorophenoxy)-3-((3-methoxy-4-(2-(4-methylpiperidia-1-yl)ethoxy) benzyl)(methyl)amino)propan-2-ol (CHJ03043)

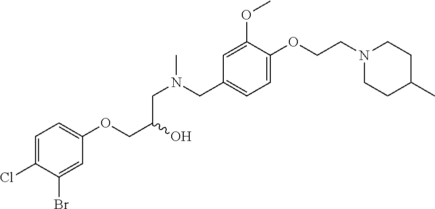 [SMILES: COc1cc(CN(C)CC(O)COc2ccc(Cl)c(Br)c2)ccc1OCCN1CCC(C)CC1]

Compound 5 used is 1-(3-methoxy4(2-4-methylpiperidin-1-yl)ethoxY)phenyl)-N-methylmethanamine (5-1),

Compound 7 used is 2-((3-bromo-4-chlorophenoxy)methyl)oxitane(7-17).

Colorless oil,yield 89%, ¹H NMR (CDCl₃, 400 MHz)δ (ppm): 730 (m, 1H), 7.17 (s, 1H), 6.81 (m 4H), 4,11 (m, 3H), 3.91 (m, 2H), 3.84 (s, 3H), 3.61 (d,J=12.0 Hz, 1H), 3.45 (d.J=12.80 Hz, 1H) 2.98 (d,J=11.20 Hz, 2H), 2.84 (t,j=6.40 Hz, 2H), 2.61 (m, 1H) 2.47 (dd,J=12.40, 3.60 Hz, 1H), 2.29 (s,3H), 2.11 (t,J=11.20 Hz, 2H), 1.63 (d,J=12.40 Hz, 2H), 1.29 (m,3H), 0.93 (d,J=6.0 Hz, 3H). ¹³CNMR(CDCl₃,100 MHz) δ (ppm): 157.74, 149.48, 147.66, 131.13, 130.47, 126.18, 122.53, 121.31, 119.57, 115.31, 113.07, 112.55, 71.00, 66.93, 65.99, 62.37, 59.10, 57.39, 55.99, 54.50 (2C), 42.29, 34.21 (2C), 30.56, 21.87IR (KBr, cm⁻¹): 2923, 2846, 2360, 2340, 1700, 1651, 1613, 1590, 1559, 1539, 1511, 1470, 1460, 1418, 1373, 1337, 1288, 1262, 1229, 1157, 1138, 1083, 1035, 931, 859, 805, 669. HRMS (ESI): m/z calcdfor C₂₆H₃₇BrClN₂O₄ (M+H)⁺: 555.1.625. found: 555.1610.

### EMBODIMENT 8

synthesis of 1-(3-bromo-4-methylphenoxy)-3-((3-methoxy-4-(2-(4-methylpiperidin-1-yl)ethoxy) benzyl)(methyl)amino)propan-2-ol (CHJ04010)

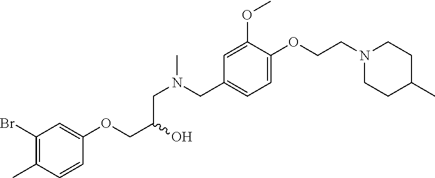 [SMILES: COc1cc(CN(C)CC(O)COc2ccc(C)c(Br)c2)ccc1OCCN1CCC(C)CC1]

Compound 5 used is 1-(3-methoxy-4-(2-(4-methylpiperidin-1-yl)ethoxy)phenyl)-N-methylmethanamine (5-1),

Compound 7 used is 2-((3-bromo-4-methylphenoxy)methyl)oxirane (7-18).

Colorless oil,yield 82%, ¹H NMR (CD₃OD, 400 MHZ)δ (ppm): 7.15 (d,J=8.40 Hz, 1H), 7.09 (s, 1H), 6.96 (s, 1H), 6.82 (m, 3H), 4.11 (t,J=5.60 Hz, 2H), 4.04 (m, 1H), 3.95 (m, 1H), 3.83 (m, 1H) 3.77 (s, 3H), 3.51 (q,J=12.80 Hz, 2H), 3.03 (d, J=11.60 Hz, 2H), 2.80 (t,J=5.60 Hz, 2H), 2.60 (dd,J=12.40, 5.60Hz, 1H), 2.48 (dd,J=13.20, 6.80 Hz, 1H), 2.30 (d,J=2.40 Hz, 6H), 2.17 (t, J=11.60 Hz, 2H.), 1.65 (d,J=12.40 Hz, 2H), 1.40 (s, 1H), 1.27 (m, 2H), 0.94 (d, J=6.40 Hz, 3H). ¹³CNMR(CD₃OD,100 MHz)δ (ppm): 157.77, 149.67, 147.44, 131.94, 130.79, 129.42, 124.18, 121.46, 117.97, 113.65, 113.61, 113.06, 70.79, 67.42, 66.75, 62.25, 59.98, 57.13, 55.00, 53.99 (2C), 42.14, 33.47 (2C), 30.24. 20.74, 20,50.1IR (KBr, cm⁻1): 2946, 2923, 2871, 2792, 2360, 2340, 1651, 1604, 1579, 1539, 1511, 1492, 1458, 1418, 1368, 1323, 1289, 1262, 1236, 1158, 1138, 1086, 1030, 1003, 932, 866, 838, 806, 757, 671. HRMS (ESI); m/zcalcd for C₂₇H₄₀BrN₂O_{O} (M+H)⁺: 535.2171. found: 535.2149.

### EMBODIMENT 19

synthesis of 1-(2-bromo-5-(trifluoromethyl)phenoxy)-3-((3-methoxy-4-(2-(4-methylpiperidin-1-yl) ethoxy)benzyl)methyl)amino)propan-2-ol (CHJ04011)

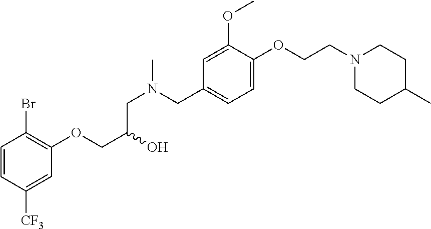 [SMILES: COc1cc(CN(C)CC(O)COc2cc(C(F)(F)F)ccc2Br)ccc1OCCN1CCC(C)CC1]

Compound 5 used is 1-(3-methoxy-4-(2-(4-methylpiperidin1-yl)ethoxy)phenyl)-N-methylmethanamine (5-1).

Compound 7 used is 2-((2-bromo-5-(trifluoromethyl)phenoxy)methyl)oxirane (7-19).

Colorless oil,yield 85%,¹H NMR (CD₃OD 400 MHz)δ (ppm): 7.71 (d,J=8.0 Hz, 1H), 7.27 (s, 1H), 7.16 (d,J=8.40 Hz, 1H), 6.95 (s, 1H), 6.82 (q,J=8.0 Hz, 3H), 4.09 (m, 5H), 3.74 (s, 3H), 3.53 (s, 2H), 3.04 (d,J=11.20 Hz, 2H), 2.81 (t,J=5.60 Hz, 2H), 2.72 (dd,J=12.40, 4.80 Hz, 1H), (dd,J=12.80, 6.40 Hz, 1H), 2.33 (s, 3H), 2.18 (t,J=12.0 Hz, 2H), 1.66 (d,J=12.80 Hz, 2H), 1.40 (s, 1H), 1.29 (m 2H), 0.94 (d,J=6.0 Hz, 3H). ¹³CNMR(CD₃OD,100 MHz)δ (ppm): 155.81, 149.65, 147.39, 133.69, 131.95, 121.42, 118.16, 118.12, 116.09, 113.57, 112.96, 109.59, 109.55, 71.58, 67.30, 66.67, 62.39, 58.82, 57.10, 54.91, 53.97 (2C), 42.13, 33.45 (2C), 30.22, 20.73. IR (KBr, cm⁻¹): 3560, 3354, 2927, 2868, 2818, 1591, 1516, 1462, 1421, 1371, 1332, 1255, 1226, 1165, 1130, 1080, 1041, 1020, 935, 904, 862, 802, 752. HRMS (ESI): m/z calcdfor C₂₇H₃₇BrF₃N₂O₄ (M+H)⁺: 589.1889. found: 589.18.27.

### EMBODIMENT 20

synthesis of 1-(3,5-dichlorophenoxy)-3-((3-methoxy-4-(2-(4-methylpiperidin-1-yl)ethoxy)benzyl) (methyl)amino)propan-2-ol (CHJ04012)

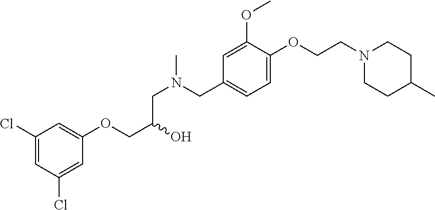 [SMILES: COc1cc(CN(C)CC(O)COc2cc(Cl)cc(Cl)c2)ccc1OCCN1CCC(C)CC1]

Compound 5 used is 1-(3-methoxy-4-(2-(4-methylpiperidin-1-yl)ethoxy)phenyl)-N-methylmethanamine (5-1).

Compound 7 used is 2-((3,5-dichlorophenoxy)methyl)oxirane (7-20).

Colorless oil,yield 88%, ¹H NMR (CD₃OD, 400 MHz)δ (ppm): 6.96 (d,J=9.60 Hz, 2H), 6.84 (m, 4H), 4.10 (t, J=5.60 Hz, 2H), 4.02 (m, 2H), 3,87 (m, 1H), 3.78 (s, 3H). 3.48 (m, 2H), 3.03 (d,J=11.20 Hz, 2H), 2.80 (t, J=6.40 Hz, 2H), 2.60 (dd,J=12.80, 6.0 Hz, 1H), 2.46 (dd,J=12.806.40 HZ,1H), 2,31 (s,3H), 2.17 (t,J=12.0 Hz, 2H), 1.65 (d, J=12.75 Hz, 2H), 1.40 (s, 1H), 1.27 (m, 2H), 0.94 (d, J=6,40 Hz, 3H). ¹³CNMR (CD₃OD, 100 MHz)δ (ppm): 160.34, 149.66, 147.46, 135.12, 131.94, 121.46, 120.38, 113.52, 113.42 (3C), 113.04, 70.04, 67.26, 66.70, 62.29, 58.68, 57.13, 54.99, 54.00 (2C), 42.25, 33.47 (2C), 30.24, 20.75. IR(KBr, cm⁻¹): 2948, 2925, 2872, 2843, 2793, 2360, 2340, 1590, 1571, 1513, 1442, 1424, 1368, 13.23, 1303, 1262, 1192, 1157, 1138, 1039, 980, 938, 853, 831, 800, 756, 670. HRMS (ESI): m/z calcd for C₂₆H₃₇Cl₂N₂O₄ (M+H)⁺: 511.2130. found: 511.2075.

### EMBODIMENT 21

synthesis of 1-(3-bromo-4-fluorophenoxy)-3-((3-methoxy-4-(2-(4-methylpiperidin-1-yl)ethoxy) benzyl)(methyl)amino)propan-2-ol (CHJ04020)

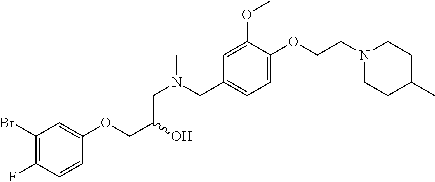 [SMILES: COc1cc(CN(C)CC(O)COc2ccc(F)c(Br)c2)ccc1OCCN1CCC(C)CC1]

Compound 5 used is 1-(3-methoxy-4-(2-(4-methylpiperidin-1-yl)ethoxy)phenyl)-N-methylmethanamine (5-1).

Compound 7 used is 2-((3-bromo-4-fluorophenoxy)methyl)oxirane (7-21).

Colorless oil,yield 90%, ¹H NMR (CD₃ODD, 400 MHz)δ (ppm): 7.10 (m, 2H), 6.95 (s, 1H), 6.84 (m, 3H), 4.10 (m, 3H), 3.96 (m, 1H), 3.84 (m, 1H), 3.77 (s, 3H), 3.50 (q,J=12.0 Hz, 2H), 3.03 (d,=11.20 Hz, 2H), 2.80 (t,J=5.20 Hz, 2H), 2.60 (dd,J=12.80, 6.0 Hz, 1H), 2.47 (dd,J=11.20, 6.80 Hz, 1H), 2.31 (s, 3H), 2.17 (t,J=11.60 Hz, 2H), 1,65 (d, 12.40 Hz, 2H), 1.41(s, 1H), 1,28 (m, 2H), 0.93 (d, J=6.0 Hz, 3H). ¹³C NMR(CD₃OD,100 MHz)δ (ppm): 155.71, 149.65, 147.44, 131.92, 121.47, 118.73, 116.32, 116.08, 114.95, 114.88, 113.55, 113.07, 71.29, 67.38, 66.71, 62.27, 58.86, 57.12, 55.01, 54.00 (2C). 42.21, 33.47 (2C), 30.23, 20.76. IR (KBr, cm⁻¹): 2947, 2925, 2872, 2843, 2793, 2360, 2340, 1591, 1513, 1493, 1458, 1418, 1368, 1322, 1262, 1220, 1203, 1157, 1138, 1088, 1035, 979, 938, 862, 840, 806, 774. HRMS (ESI); calcdfor C₂₆H₃₇BrFN₂O₄ (M+H)⁺: 539.1921. found: 539.1888.

### EMBODIMENT 22

synthesis of 1-((3-methoxy-4-(2-(4-methylpiperidin-1-yl)ethoxy)benzyl)(methy)amino)-3-(2,4,6-tribromnophenoxy)propan-2-ol (CHJ04022)

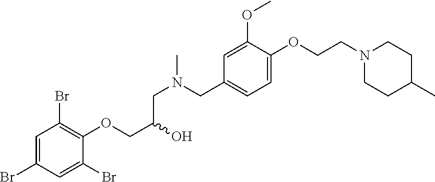 [SMILES: COc1cc(CN(C)CC(O)COc2c(Br)cc(Br)cc2Br)ccc1OCCN1CCC(C)CC1]

Compound 5 used is 1-(3-methoxy-4-(2-(4-methylpiperidin1-yl)ethoxy)phenyl)-N-methylmethanamine (5-1).

Compound 7 used is 2-((2.4,6-tribromophenoxy)metbyl)oxirane(7-22.

Colorless oii,yieid 90%, ¹H NMR. (CD₃OD, 400 MHz)δ (ppm): 7.75 (s, 2H), 6.98 (s, 1H), 6.85 (m, 2H), 4.10 (t,J=5.69 Hz, 2H), 4.22 (m, 1H), 4.12 (t,J=5.61 Hz, 2H), 3.79 (s, 3H), 3.54 (q, J=12.80 Hz, 2H), 3.03 (d, J=11.20 Hz, 2H), 2.80 (t, j=5.60 Hz, 2H), 2.72 dd, J=13.20, 5.20 Hz, 1H), 2.54 (dd, J=12.80, 7.20 Hz, 1H), 2.32 3H), (s, 3H), 2.16 (t, J=11.60 Hz 2H), 1.65 (d, J=12.40 Hz, 2H), 1.39 (s, 1H), 1.30 (m, 2H), 0.93 (d, J=6.40 Hz, 3H) ¹³CNMR(CD₃OD,100 MHz)δ (ppm): 152.75, 149.67, 147.45, 134.96 (3C), 131.75, 121.55, 118.54, 117.13, 113.66, 113.14, 75.83, 67.96, 66.75, 62.14, 59.34, 57.11, 55.06, 53.99 (2C), 42.08, 33.48 (2C), 30.24, 20.77. IR (KBr, cm⁻¹): 2923, 2846 2360, 2340, 1700, 1651, 1613, 1590, 1559, 1539, 1511, 1470,1460, 1418, 1373 1337, 1288, 1262, 1229, 1157, 1138, 1083, 1035, 931, 859, 805. HRMS (ESI): m/z calcd for C₂₆H₃₆Br₃N₂O₄ (M+H)⁺: 677.0225. found: 677.0256.

### EMBODIMENT 23

synthesis of 1-(3-bromo-5-fluorophenoxy)-3-((3-methoxy-4-(2-(4-methylpiperidin-1-yl) benzyl)(methyl)amino)propan-2-ol (CHJ04023)

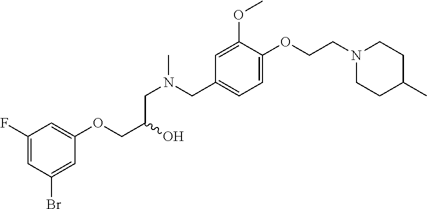 [SMILES: COc1cc(CN(C)CC(O)COc2cc(F)cc(Br)c2)ccc1OCCN1CCC(C)CC1]

Compound 5 used is 1-(3-methoxy-4-(2-(4-methylpiperidin1-yl)ethoxy)phenyl)-N-methylmethanamine (5-1).

Compound 7 used is 2-((3-bromo-5-fluorophenoxy)metbyl)oxirane(7-23 .

Colorless oil,yield 85%, ¹H NMR (CD₃OD, 400 MHz)δ (ppm): 6.95 (s, 1H), 6.85 (m, 4H), 6.65 (d, J=10.80 Hz, 1H), 4.11 (t, J=5.60 Hz, 2H), 4.02 (m, 2H), 3.87 (m, 1H), 3.78 (s, 3H), 3.50 (q, J=12.80 Hz, 2H), 3.03 (d, j=11.20 Hz, 2H), 2.81 (t, 5.60 Hz, 2H), 2.60 (dd,J=12.40, 5.60 Hz, 1H), 2.46 (dd, J=12.80, 6.40 Hz, 1H), 2.31 (s, 3H), 2.17 (t, j=12.0 Hz, 2H), 1.65 (d, J=12.80 Hz, 2H), 1.40 (s, 1H), 1.29 (m, 2H), 0.93 (d, J=6.0 Hz, 3H). ¹³CNMR(CD₃OD,100 MHz)δ (ppm): 164.64, 162.18, 161.02, 149.65, 147.45, 131.93, 122.34, 121.46, 113.83, 113.29, 110.85, 101.17, 71.09, 67.25, 66.68, 62.28, 58.72, 57.12, 55.00, 53.99 (2C), 42.23, 33.46 (2C), 30.23, 20.76. IR (KBr, cm⁻¹): 2947, 2925, 2872, 2841, 2792, 2360, 2340, 1605, 1583, 1512, 1454, 1418, 1367, 1318, 1280, 1263, 1231, 1280, 1263, 1231, 1146, 1084, 1039, 980, 941, 833. HRMS (ESI): m/z calcdfor C₂₆H₃₇BrFN₂O₄ (M+H)⁺: 539.1921. found:539.1879.

### EMBODIMENT 24

synthesis of 1-(3-chlorophenoxyl-3-((3-methoxy-4-(2-(4-methylpiperidin-1-yl)ethoxy)benzyl) (methyl)amino)propan-2-ol (CHJ04024)

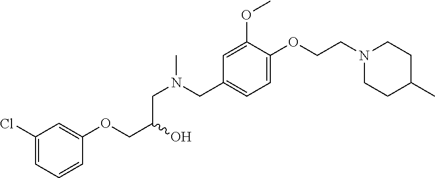 [SMILES: COc1cc(CN(C)CC(O)COc2cccc(Cl)c2)ccc1OCCN1CCC(C)CC1]

Compound 5 used is 1-(3-methoxy-4-(2-(4-methylpiperidin-1-yl)ethoxy)phenyl)-N-methylmethanamine (51).

Compound 7 used is 2-((3-chlorophenoxy)methyl)oxirane (7-24).

Colorless oil,yield 83%, ¹H NMR (CD₃OD, 400 MHz)δ (ppm): 7.21 (t,J=8.40 Hz, 1H), 6.88 (m, 6H), 4.09 (m, 3H), 3.98 (m, 1H), 3.86 (m, 1H), 3.77 (s, 3.51 (q, J=12.80 Hz, 1H), 3.03 (d,J=11.20 Hz, 1H), 2.80 (t, J=5.60 Hz, 2H), 2.61 (dd, J=12.80, 6.80 Hz, 2H), 2.49 (dd, J=12.80, 6.80 Hz, 2H), 2.30(s, 3H), 2.16 (t, J=11.60 Hz, 2H), 1.65 (d, J=12.80 Hz, 2H), 1.39 (s, 1H), 1.28 (m, 2H), 0.93 (d, J=6.0 Hz, 3H), ¹³CNMR (CD₃OD, 100 MHz)δ (ppm): 159.91, 149.66, 147.45, 134.44, 131.93, 130.12, 121.47, 120.46, 114.57, 113.58, 113.06, 112.81, 70.71, 67.38, 66.72, 62.26, 58.98, 57.13, 55.00, 53.99 (2C), 42.16, 33.48 (2C), 30.24, 20.76. IR (KBr, cm⁻¹): 2947, 2925, 2872, 2843, 2792, 2360, 2340, 1651, 1595, 1580, 1539, 1511, 1470, 1459, 1419, 1367, 1326, 1283, 1260, 1231, 1192, 1157, 1138, 1091, 1036, 979, 935, 870, 807, 770, 681, HRMS (ESI): m/z calcdfor C₂₆H₃₈ClN₂O₄ (M+H)⁺: 477.2520. found; 477.2476.

### EMBODIMENT 25

synthesis of 1-((3-methoxy-4-(2-(4-methylpiperidin-1-yl)ethoxy)benzyl)(methyl)amino)-3-(3-(trifluoromethyl)phenoxy)propan-2-ol (CHJ04025)

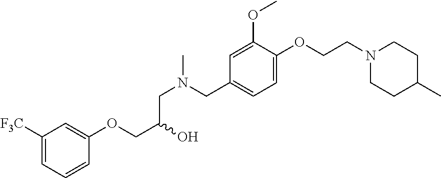 [SMILES: COc1cc(CN(C)CC(O)COc2cccc(C(F)(F)F)c2)ccc1OCCN1CCC(C)CC1]

Compound 5 used is 1-(3-methoxy-4-(2-(4-methylpiperidin-1-yl)ethoxy)phenyl)-N-methylmethanamine (51).

Compound 7 used is 2-((3-(trifluoromethyl)phenoxy)methyl)oxirane (7-25).

Colorless oil,yield 83%, ¹H NMR (CD₃OD, 400 MHz)δ (ppm): 7.44 (t,J=8.0 Hz, 1H), 7.17 (m, 3H), 6.97 (s, 1H), 6.85 (m, 2H), 4.09 (m, 4H), 3.93 (m, 1H), 3.76 (s, 3H), 3.53 (m,, 2H), 3.03 (d, J=11.20 Hz, 2H), 2.80 (t, J=5.60 Hz, 2H), 2.63 (dd, J=12.40, 5.20 Hz, 1H), 2.51 (dd, J=12.80, 6.80 Hz, 1H), 2.31 (s, 3H), 2.17 (t, J=11.60 Hz, 2H), 1.65 (d, J=12.80 Hz, 2H), 1.40 (s, 1H), 1.28 (m, 2H), 0.93(d, J=6.40 Hz, 3H), ¹³CNMR (CD₃OD, 100 MHz) δ (ppm): 159.32, 149.66, 147.45, 131.92, 131.39, 131.55, 130.01, 121.46, 117.96, 116.91, 113.57, 113.06, 110.96, 70.71, 67.38, 66.72, 62.26, 58.98, 57.13, 55.00, 53.99 (2C), 42.16, 33.48 (2C), 30.24, 20.76. IR (KBr, cm⁻¹): 2947, 2926, 2873, 2845, 2794, 2360 2340, 1651, 1593, 1557, 1539, 1513, 1493, 1453, 1419, 1367, 1330, 1289, 1262, 1234, 1165, 1096, 1065, 1037, 979,934, 880, 794, 753, 698. HRMS (ESI): m/z calcdfor C₂₇H₃₈F₃N₂O₄ (M+H)⁺: 511.2784. found: 511.2765.

### EMBODIMENT 26

synthesis of 1-(3,4-dichlophenoxy)-3-((3-methoxy-4-(2-(4-methylpiperidin-1-yl)ethoxy)benzyl) (methyl)amino)propan-2-ol (CHJ04026)

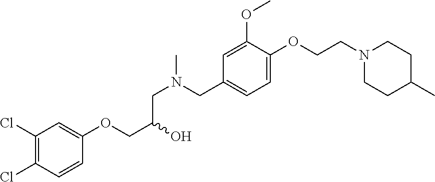 [SMILES: COc1cc(CN(C)CC(O)COc2ccc(Cl)c(Cl)c2)ccc1OCCN1CCC(C)CC1]

Compound 5 used is 1-(3-methoxy-4-(2-(4-methylpiperidin-1-yl)ethoxy)phenyl)-N-methylmethanamine (51).

Compound 7 used is 2-((3,4-dichlorophenoxy)methyl)oxirane (7-26).

Colorless oil,yield 81%, ¹H NMR (CD₃OD, 400 MHz)δ (ppm): 7.36 (d,J=9.20 Hz, 1H), 7.05 (s, 1H), 6.95 (s, 1H), 6.84 (m, 3H), 4.10 (t,J=5.60 Hz, 2H), 4.04 (m, 1H), 3.97 (m, 1H), 3.86 (m, 1H), 3.77 (s, 3H), 3.50 (q,J=12.80 Hz, 1H ), 3.03 (d,J=11.60 Hz, 2H), 2.80 (t,J=5.60 Hz, 2H), 2.61 (dd,J=12.80, 6.0 Hz, 1H), 2.47 (dd, J=12.80, 6.80 Hz1H), 2.31 (s, 3H), 2.16 (t,J=11.60 Hz, 2H), 1.65 (d,J=12.80 Hz, 2H), 1.40 (s, 1H), 1.27 (m, 2H), 0.94 (d, J=6.40 Hz, 3H). ¹³C NMR (CD₃OD, 100 MHz)δ (ppm); 158.36, 149.64, 147.45, 132.25, 131.93, 130.48, 123.33, 121.47, 116.16, 114.57, 113.52, 113.07, 70.99, 67.33, 66.70, 62.29, 58.76, 57.13, 54.98, 54.00 (2C), 42,26, 33.48 (2C), 30.24, 20.76. IR (KBr, cm⁻¹): 2947, 2926, 2872 2841, 2792, 2360, 2340, 1651, 1592, 1570, 1539, 1511, 1475, 1455, 1419, 1367, 1286, 1262, 1230, 1191, 1156, 1127, 1092, 1036, 979, 937, 861, 807, 757, 670. HRMS (ESI): m/z calcdfor C₂₆H₃₇Cl₂N₂O₄ (M+H)⁺: 511.2130. found: 511.2115.

### EMBODIMENT 27

synthesis of 1-(2-iodophenoxy)-3-((3-methoxy-4-(2-(4-methylpiperidin-1-yl)ethoxy)benzyp(methyl)amino)propan-2-ol (CHJ04027)

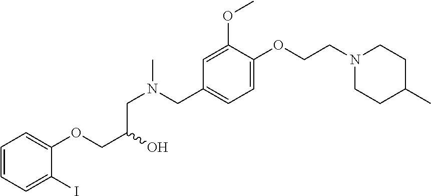 [SMILES: COc1cc(CN(C)CC(O)COc2ccccc2I)ccc1OCCN1CCC(C)CC1]

Compound 5 used is 1-(3-methoxy-4-(2-(4-methylpiperidin-1-yl)ethoxy)phenyl)-N-methylmethanamine (51).

Compound 7 used is 2-((2-iodophenoxy)methyl)oxirane (7-27).

Colorless oil,yield 90%,¹H NMR (CD₃OD, 400 MHz)δ (ppm): 7.72 (d,J=7.60 Hz, 1H) 7.30 (t,J=8.0 Hz, 1H), 6.96 (s,1H), 6.90 (d,J=8.40 Hz, 1H), 6.83 (q,J=8.0 Hz, 2H), 6.70 (t,J=7.60 Hz, 1H), 4.09 (m, 3H), 3.98 (m,2H), 3.74 (s, 3H), 3.55 (q, J=12.80 Hz, 2H), 3.03 (d,J=11.20 Hz, 2H), 2.80 (t,J=5.60 Hz), 2H), 2.74 (d, J=5.20 Hz, 1H), 2.64 (dd,J=12.80, 7.60 Hz, 1H), 2.32 (s, 3H), 2.17 (t, J=11.60 Hz, 2H), 1.65 (d, 12.80 Hz, 2H), 1.40(s, 1H), 1,29 (m, 2H), 0.94 (d, J=6.40 Hz, 3H) ¹³CNMR (CD₃OD, 100 MHz)δ (ppm): 157.53, 149.66, 147.40, 139.11, 131.94, 129.30, 122.32, 121.47, 113.60, 113.07, 112.12, 85.66, 71.22, 67.40, 62.42, 59.28, 57.11, 55.00, 53.97 (3C), 42.07, 33.47 (2C), 30.23, 20.75 IR (KBr, cm⁻¹): 2946, 2923, 2871, 2844, 2792, 2360, 2340, 1584, 1513, 1471, 1441, 1418, 1368, 1323, 1261, 1231, 1192, 1158, 1138, 1084, 1050, 1030, 1019, 979, 962, 938, 873, 822, 806, 749. HRMS (ESI): m/z calcdfor C₂₆H₃₈IN₂O₄ (M+H)⁺: 569.1876. found: 569.1842.

### EMBODIMENT 28

synthesis of 1-(4-bromo-2,6-dichlorophenoxy)-3-((3-methoxy-4-(2-(4-methylpiperidin-1-yl)ethoxy)benzyl)(methyl)amino)propan-2-ol (CHJ04033)

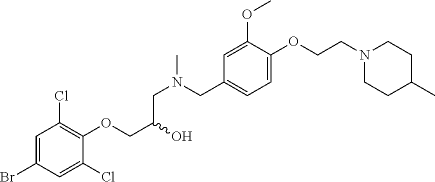 [SMILES: COc1cc(CN(C)CC(O)COc2c(Cl)cc(Br)cc2Cl)ccc1OCCN1CCC(C)CC1]

Compound 5 used is 1-(3-methoxy-4-(2-(4-methylpiperidin-1-yl)ethoxy)phenyl)-N-methylmethanamine (51).

Compound 7 used is 2-((4-bromo-2,6-dichlorophenoxy)methyl)oxirane (7-28).

Colorless oil,yield 84%, ¹H NMR (CD₃OD, 400 MHz)δ (ppm): 7.56 (s, 2H), 6.97 (s, 1H), 6.85 (m, 2H), 4.16 (m, 1H), 4.12 (t, J=5.60 Hz, 2H), 3.99 (m, 2H), 3.79 (s, 3H), 3.52 (m, 2H), 3.04 (d, J=11.20 Hz, 2H), 2.81 (t, J=5.60 Hz, 2H), 2.67 (dd, J=13.20, 4.80 Hz 1H), 2.55 (dd, J=13.20, 7.60 Hz, 1H ) 2.30 (s, 3H), 2.17 (t, J=12.0 Hz, 2H), 1.65 (d, J=12.40 Hz, 2H) 1.40 (s, 1H) 1.29 (m, 2H), 0.94 (d, J=6.0 Hz, 3H), ¹³CNMR (CD₃OD, 100 MHz)δ (ppm): 150.93, 149.67, 147.43, 131.79, 131.46 (3C), 129.99, 121.50, 116.13, 113.67, 113.08, 76.07, 67.97, 66.71, 62,14, 59.26, 57.09, 55.02. 53.97 (2C), 42.01, 33.44 (2C), 30.21, 20,.76. IR. (KBr, cm⁻¹): 2947, 2924, 2872, 2840, 2794, 2360, 2340, 1544, 1511, 1459, 1419, 1375, 1320, 1259, 1231, 1193, 1158, 1138, 1084, 1031, 994, 933, 856, 803. HRMS (ESI): for C₂₆H₃₆BrCl₂N₂O₄ (M+H)⁺: 589.1236. found: 589.1220.

### EMBODIMENT 29

synthesis of 1-(3-bromo-5-chlorophenoxy)-3-((3-methoxy-4-(2-(4-methylpiperidin1-yl)ethoxy) benzyl)(methyl)amino)propan-2-ol. (CHJ04034)

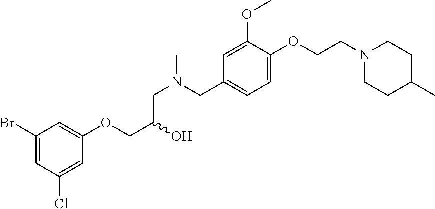 [SMILES: COc1cc(CN(C)CC(O)COc2cc(Cl)cc(Br)c2)ccc1OCCN1CCC(C)CC1]

Compound 5 used is 1-(3-methoxy-4-(2-(4-methylpiperidin-1-yl)ethoxy)phenyl)-N-methylmethanamine (51).

Compound 7 used is 2-((3-bromo-5-chlorophenoxy)methyl)oxirane (7-29).

Colorless oil,yield 85%,¹H NMR (CD₃OD, 400 MHz)δ (ppm): 7.12 (s, 1H), 7.02 , 6.95 (s, 1H ), 6.91 (s, 1H), 6.83 (m, 2H), 4.10 5.60 Hz, 2H), 4.02 (m, 2H), 3.87 (m, 1H ), 3.78(s, 3H), 3.48 (m, 2H), 3.04 (d,J=11.60 Hz, 2H), 2.81 (t,J=5.60 Hz, 2H), 2.60 (dd,J=12.40, 6.0 G), 2,46 (dd,J=12.40, 6.0 Hz, 1H), 2.31 (s, 3H), 2.18 (t,J=12.0 Hz, 2H), 1.66 (d,J=12.80 Hz, 2H), 1.40 (s, 1H), 1.27 (m, 2H), 0.94 (d,J=6.0 Hz, 3H). ¹³CNMR(CD₃OD, 100 MHz)δ (ppm): 160.39, 149.66, 147.44, 135.27, 131.85, 123.18, 122.49, 121.46, 116.33, 113.87, 113.54, 113.04, 71.04, 67.26, 66.67, 62.29, 58.67, 57.11, 55.01, 53.99 (2C), 42.26, 33.45 (2C), 30.22, 20.74, IR (KBr, cm⁻¹): 2947, 2926, 2870, 2840, 2793, 2360, 2331, 1588, 1563, 1539, 1512, 1459, 1437, 1420, 1367, 1335, 1319, 1301, 1230, 1259, 1190, 1156, 1138, 1091, 1038, 978, 930, 912, 864, 831, 770, 670, HRMS (ESI): m/z calcd for C₂₆H₃₇BrClN₂O₄ (M+H)⁺: 555.1625. found: 555.1600.

### EMBODIMENT 30

synthesis of 1-(2-bromo-5-fluorophenoxy)-3-((3-methoxy-4-(2-(4-methylpiperidin-1-yl)ethoxybenzyl)(methyl)amino))propan-2-ol (CHJ04036)

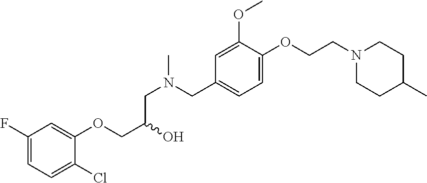 [SMILES: COc1cc(CN(C)CC(O)COc2cc(F)ccc2Cl)ccc1OCCN1CCC(C)CC1]

Compound 5 used is 1-(3-methoxy-4-(2-(4-methylpiperidin-1-yl)ethoxy)phenyl)-N-methylmethanamine (51).

Compound 7 used is 2-((2-bromo-5-fluorophenoxy)methyl)oxirane (7-30).

Colorless oil,yield 82%, ¹H NMR (CD₃OD, 400 MHz)δ (ppm): 7.48 (m, 1H), 6.95 (s, 1H), 6.82 (m, 3H), 6.63 (t,J=8.40 Hz, 1H), 4.11 (t,J=5.6 Hz, 3H), 4.01 (m, 2H), 3.76 (s, 3H), 3.53 (s, 2H), 3.04 (d,J=11.20 Hz, 2H), 2.81 (t, J=5.6 Hz, 2H), 2.71 (dd,J=12.80, 5.20 Hz, 1H), 2.58 (dd, J=12.80, 7.20 Hz, 1H), 2.32 (s, 3H), 2.18 (t, J=11.60 Hz, 2H), 1.66 (d, J=12.80 Hz, 2H), 1.41 (s, 1H), 1.29 (m, 2H ), 0.94 (d J=6.40 Hz. 3H). ¹³CNMR (CD₃OD, 100 MHz)δ (ppm): 164.01, 161.58, 156.35, 149.65, 147.40, 133.34, 131.93, 121.47, 113.59, 107.89, 106.05, 101.44, 71.52, 67.28, 66.65, 62.36, 58.95, 57.09, 54.96, 53.96 (2C), 42.01, 33.43 (2C), 30.21, 20.73. IR (KBr, cm⁻¹): 3529, 3277, 3088, 2929, 2852, 2796, 2769, 2428, 1681, 1604, 1514, 1477, 1452, 1417, 1371, 1286, 1259, 1224, 1151, 1101, 1037, 960, 871, 833, 790, 748, 609, 451. HRMS (ESI): m/z calcd for C₂₆H₃₇BrFN₂O₄ (M+H)⁺: 539.1921. found: 539.1914.

### EMBODIMENT 31

synthesis of N-(3-(2-hydroxy-3((3-methoxy-4-(2-(4-methylpiperidin-1-yl)ethoxy)benzyl)(methyl)amino)propoxy)phenyl)acetamide (CHJ04058)

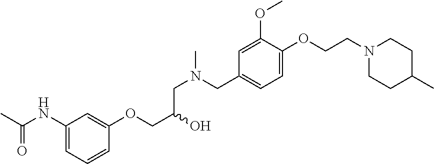 [SMILES: COc1cc(CN(C)CC(O)COc2cccc(NC(C)=O)c2)ccc1OCCN1CCC(C)CC1]

Compound 5 used is 1-(3-methoxy-4-(2-(4-methylpiperidin-1-yl)ethoxy)phenyl)-N-methylmethanamine (51).

Compound 7 used is N-(3-oxirane-2-ylmethoxy)phenyl)acetamide (7-31).

White solid,yield 90%,mp: 60-62° C.,¹H NMR (CD₃OD, 400 MHz)δ (ppm): 7.27 (s, 1H), 7.17 (t,,J=8.0 Hz, 1H), 7.05 (m, 2H), 6.86 (m 2H), 6.63 (d, J=8.0 Hz, 1H), 4.11 (m, 3H), 3.97 (m, 1H), 3.87 (m, 1H), 3.78 (s, 3H), 3.55 (s, 2H), 3.13 (dd,J =11.20 Hz, 2H), 2.91 (t,J=5.20 Hz, 1H ), 2.65 (dd,J=12.40, 6.80 Hz, 1H), 2.53 (dd,J=12.40, 6.80 Hz,1H ), 2.32 (m, 5H), 2.11 (s, 3H), 1.70 (d, J=13.20 Hz, 2H), 1.46 (s, 1H ), 1.32 (m, 3H), 0.94 (d,J=6.0 Hz, 3H), ¹³CNMR (CD₁OD, 100 MHz)δ (ppm): 170.23, 159.35, 149.61, 147.25, 139.66, 131.87, 129.11, 121.58, 113.73, 113.06, 112.11, 109.83, 106.29, 70.42, 67.37, 66.13, 62.12, 59.09, 56.86, 55.00, 53.79 (2C), 42.01, 33.02 (2C), 29.92, 22.53, 20.59, IR (KBr, cm−1); 2924, 2852, 2360, 2340, 1699, 1670, 1651, 1616, 1556, 1540, 1510, 1491, 1458, 1419, 1373, 1286, 1265, 1230, 1198, 1156, 1083, 1034, 980, 871, 768, 686, 669. HRMS (ESI): m/z calcd for C₂₈H₄₂N₃O₅ (M+H)⁺: 500.3124. found: 500.3071.

### EMBODIMENT 32

synthesis of 1-(2,4-dichlorophenoxy)-3-((3-methoxy-4-(2-(4-methylpiperidin-1-yl)ethoxy)benzyl) (methyl)amino)propan-2-ol (CHJ04059)

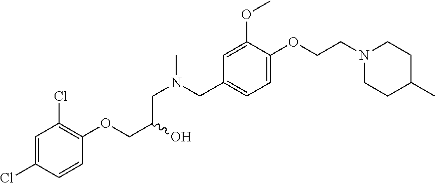 [SMILES: COc1cc(CN(C)CC(O)COc2ccc(Cl)cc2Cl)ccc1OCCN1CCC(C)CC1]

Compound 5 used is 1-(3-methoxy-4-(2-(4-methylpiperidin-1-yl)ethoxy)phenyl)-N-methylmethanamine (51).

Compound 7 used is 2-((2,4-dichlorophenoxy)methyl)oxirane (7-32).

Colorless oil,yield 81%,¹H NMR. (CD₃OD, 400 MHz)δ (ppm): 7.37 (s, 1H ), 7.23 (d,J=8.40 Hz, 1H), 7.00 (d,J=8.80 Hz, 1H), 6.94 (s, 1H), 6.82 (q,J=8.0 Hz, 2H), 4.11 (t,J=5.60 Hz, 3H), 4.00 (m, 2H), 3.75 (s, 3H), 3.52 (s. 2H), 3.06 (d,J=11.60 Hz, 2H), 2.82 (t,J=5.60 Hz, 2H), 2.69 (dd,J=12.80, 5.60 Hz, 1H), 2.54 (dd,J=12.80, 6.80 Hz, 1H), 2.32 (s, 3H), 2.19 (tmJ=12.0 Hz, 2H), 1.67 (d,J=12.80 Hz, 2H ) (s,1H ), 1.29 (m, 2H), 0.94 (d,J=6.40 Hz, 3H). ¹³CNMR(CD₃OD, 100 MHz)δ (ppm): 153.47, 149.61, 147.37, 131.88, 129.29, 127.46, 125.32, 123.37, 121.45, 114.33, 113.50, 112.98, 71.53, 67.36, 66.58, 62.33, 58.86, 57.07, 54.92, 53.96 (2C), 42.19, 33.41 (2C), 30.20, 20.73. IR (KBr, cm−1): 2947, 2924, 2872, 2845, 2360, 2339, 1590, 1513, 1484, 1458, 1419, 1389, 1368, 1323, 1290, 1263, 1232, 1156, 1060, 1028, 1007, 938, 867, 846, 804, 745, 653, HRMS (ESI): calcd for C₂₆H₃₇Cl₂N₂O₄ (M+H)⁺: 511.2130, found: 511.2120.

### EMBODIMENT 33

synthesis of 1-(5-bromo-2-methylphenoxy)-3-((3-methoxy-4-(2-(4-methylpiperidin-1-yl)ethoxy)benzyl)(methyl)amino)propan-2-ol (CHJ04060)

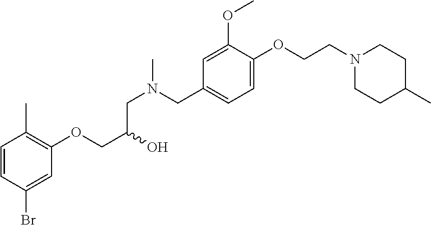 [SMILES: COc1cc(CN(C)CC(O)COc2cc(Br)ccc2C)ccc1OCCN1CCC(C)CC1]

Compound 5 used is 1-(3-methoxy-4-(2-(4-methylpiperidin-1-yl)ethoxy)phenyl)-N-methylmethanamine (51).

Compound 7 used is 2-((5-bromo-2-methylphenoxy)methyl)oxirane (7-33).

Colorless oil,yield 82%. 1H NMR (CD₃OD, 400 MHz)δ (ppm): 6.97 (m, 4H), 6.82 (q, j=8.0 Hz, 2H), 4.10 (t, J=5.20 Hz, 3H), 3.97 (dd,J=9.60, 2.80 Hz, 1H), 3.89 (dd,J=9.20, 5.2Hz,1H), 3.74 (s, 3H), 3.51 (q,J=12.8 Hz, 2H), 3.04 (d, J=11.20 Hz, 2H), 2.81 (t, J=5.20 Hz, 2H), 2.66 (dd, J=12.80, 6.0 Hz, 1H), 2.49 (dd, J=12.80, 6.0 Hz, 1H), 2.33 (s, 3H), 2.18 (t, J=11.60 Hz, 2H), 2.05 (s, 3H), 1.66 (d, J=12.80 Hz, 2H), 1.41 (s, 1H), 1.27(m, 2H), 0.94 (d, J=6.0 Hz, 3H). ¹³CNMR(CD₃OD, 100 MHz)δ (ppm): 157.70, 149.65, 147.37, 131.99, 131.30, 125.78, 122.88, 121.36, 119.06, 113.96, 113.48, 112.83, 70.39, 67.47, 66.64, 62.41, 58.80, 57.10, 54.91, 53.98 (2C), 42.28, 33.45 (2C), 30.23, 20.72, 14.59, (KBr, cm⁻¹): 2947, 2923, 2871, 2844, 2360, 2340, 1592, 1555, 1513, 1491, 1458, 1417, 1398, 1373, 1260, 1239, 1191, 1129, 1084, 1034, 984, 939, 870, 836, 800, 756. HRMS (ESI): calcd for C₂₇H₄₀BrN₂O₄ (M+H)⁺: 535.2171. found: 535,2170.

### EMBODIMENT 34

synthesis of N-(4-2-hydroxy 3-((3-methoxy-4-(2-(4-methylpiperidin-1-yl)ethoxy)benzyl)(methyl)amino)propoxy)phenypacetamide (CHJ04061)

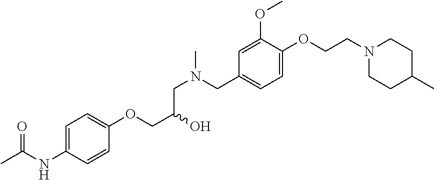 [SMILES: COc1cc(CN(C)CC(O)COc2ccc(NC(C)=O)cc2)ccc1OCCN1CCC(C)CC1]

Compound 5 used is 1-(3-methoxy-4-(2-(4-methylpiperidin-1-yl)ethoxy)phenyl)-N-methylmethanamine (51).

Compound 7 used is N-(4-(oxiran-2-ylmethoxy)phenyl)acetamide (7-34).

White solid,yield 90%,mp: 60-62° C., ¹H NMR (CD₃OD, 400 MHz)δ (ppm): 7.41 (d,J=8.0 Hz, 2H), 6.98 (s,1H ), 6.86 (m, 4H), 4.14 (m, 3M), 3.96 (m, 1H ), 3.85 (m, 1H), 3.78 (s, 3H), 3.54 (d, J=5.60 Hz, 2H), 3.14 (d,J=11.20 Hz, 2H), 2.92 (t,J=5.20 Hz, 1H ), 2.64 (dd,J=12.80, 7.20 Hz 1H), 2.52 (dd,J=12.80, 7.20 Hz,1H), 2.32 (m, 5H), 2.09 (s, 3H), 1.70 (d,J=13.20 Hz, 2H), 1.46 (s, 1H), 1.32 (m, 3H), 0.95 (d, J=6.40 Hz, 3H), ¹³CNMR (CD₃OD, 100 MHz)δ (ppm): 170.23, 159.35, 149.61, 147.25, 139.66, 131.87, 129.11, 121.58, 113.73, 113.06, 112.11, 109.83, 106.29, 70.42, 67.37, 66.13, 62.12, 59.09, 56.86, 55.00, 53.79 (2C.), 42.01, 33.02 (2C), 29.92, 22.53, 20.59, IR (KBr, cm⁻¹): 2922, 2848, 2362, 2340, 2044, 1681, 1602, 1548, 1512, 1460, 1417, 1369, 1325, 1259, 1240, 1136, 1031, 931, 819, 750, 686, 669. HRMS (ESI) m/z calcd for C₂₈H₄₂N₃O₅ (M+H)⁺: 500.3124. found: 500.3074.

### EMBODIMENT 35

synthesis of 14(3-methoxy-4-(2(4-methylpiperidin1-y;)ethoxy)benzyl)(methyl)amino)-3-(2-(trifluoromethyl)phenoxy)propan-2-ol (CHJ04082)

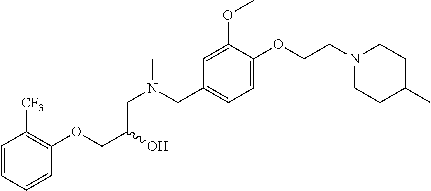 [SMILES: COc1cc(CN(C)CC(O)COc2ccccc2C(F)(F)F)ccc1OCCN1CCC(C)CC1]

Compound 5 used is 1-(3-methoxy-4-(2-(4-methylpiperidin-1-yl)ethoxy)phenyl)-N-methylmethanamine (51).

Compound 7 used is 2-((2-trifluoromethyl)phenoxy)methyl)orirane (7-35).

Colorless oil,yield 83%, ¹H NMR (CDCl₃, 400 MHz) δ (ppm): 7.55 (d, J=7.60 Hz, 1H), 7.47 (t,J=8.0 Hz, 1H), 7.00 (t,J=8.40 Hz, 2H), 6.81 (m, 3H), 4.10 (m, 5H), 3.84 (s, 3H), 3.62 (d, J=12.80 Hz, 1H), 3.47 (d, J=12.80 Hz,1H), 3.00 (d,J=10.80 Hz, 2H), 2.86 (t,J=6.0 Hz, 2H), 2.68 (m, 1H), 2.59 (m, 1H), 2.30 (s, 3H), 2.15 (t,J=10.80 Hz, 2H), 1.64 (d,J=12.0 Hz, 2H), 1.31 (m, 3H), 0.93 (d,J=5.60 Hz, 3H). ^{—}C NMR(CDCl₃,100 MHz) δ (ppm): 156.61, 149.49, 147.54, 133.28, 131.37, 127.08. 121.34 (2C), 120.33 (2C), 113.18, 112.96, 112.61, 70.86. 66.82, 66.17, 62.45, 59.40, 57.34, 55.95, 54.43 (2C), 42.43, 34.08 (2C), 30.50, 21.81. IR (KBr, cm⁻¹): 2039, 2873, 2841, 2794, 2362, 1602, 1510, 1460, 1363, 1323, 1269, 1132, 1033, 974, 948, 879, 808, 758, 650. HRMS (ESI): m/z calcd for C₂₇H₃₈F₃N₂O₄ (M+H)⁺: 511.2784. found: 511.2741.

### EMBODIMENT 36

synthesis of 1-(4-chlorophenoxy)-3((3-methoxy-4-(2-(4-methylpiperidin-1-yl)ethoxy)benzyl)(methyl)amino)propan-2-ol (CHJ04083)

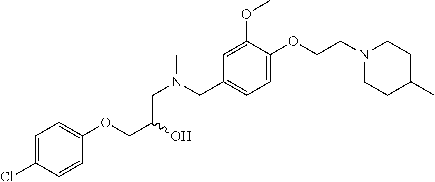 [SMILES: COc1cc(CN(C)CC(O)COc2ccc(Cl)cc2)ccc1OCCN1CCC(C)CC1]

Compound 5 used is 1-(3-methoxy-4-(2-(4-methylpiperidin-1-yl)ethoxy)phenyl)-N-methylmethanamine (51).

Compound 7 used is 2-((4-methoxyphenoxy)methyl)oxirane (7-36).

Colorless oil,yield 90%, ¹H NMR (CDCl₃, 400 MHz) δ (ppm): 7.22 (d,J=15.2 Hz, 2H), 6.81 (m, 5H), 4.16 (t,J=6.0 Hz, 2H), 4.09 (m, 1H), 3.92. (d,J=4.40 Hz, 2H), 3.84 (s, 3H), 3.62 (d, J=13.20 Hz, 1H ), 3.45 (d, J=13.20 Hz, 1H), 2.99 (d, J=10.8 Hz, 2H), 2.85 (t,J=6.0 Hz, 2H), 2.63 (t,J=11.60 Hz, 1H), 2.50 (m, 1H), 2.29 (s, 3H), 2.14 (t,J=10.80 Hz, 2H), 1.64 (d,J=12.40 Hz, 2H), 1.32 (m, 3H), 0.93 (d,J=5.60 Hz, 3H), ¹³CNMR(CDCl₃,100 MHz) δ (ppm): 157.41, 149.51, 147.6.2, 131.25, 129.30 (2C), 125.84, 121.32, 115.88 (2C), 113.16, 112.59, 70.74, 66.91, 66.11, 62.37, 59.29, 57.36, 55.99, 54.47 (2C), 42.28, 34.12 (2C), 30.52, 21.83. IR (KBr cm⁻¹): 2947, 2925, 2872, 2843, 2792, 2360, 2325, 1651, 1595, 1539, 1511, 1492, 1458, 1418, 1367, 1322, 1283, 1246, 1157, 1138, 1092, 1035, 1008, 824, 672. HRMS (ESI): calcd for C₂₆H₃₈ClN₂O₄ (M+H)⁺: 477.2520. found: 477.2471.

### EMBODIMENT 37

synthesis of 1-((3-methoxy-4-(2-(4-methylpiperidin-1-yl)ethoxy)benzyl)(methyl)amino)-3-(4-methoxyphenoxy)propan-2-ol (CHJ04084)

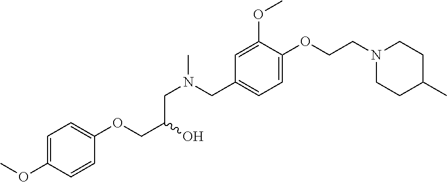

Compound 5 used is 1-(3-methoxy-4-(2-(4-methylpiperidin-1-yl)ethoxy)phenyl)-N-methylmethanamine (51).

Compound 7 used is 2-((4-methoxyphenoxylmethyl)oxirane (7-37).

Colorless oil,yield 90%, ¹HNMR (CDCl₃, 400 MHz) δ (ppm): 6.82 (m, 7H), 4.17 (t,J=6.0 HZ, 2H), 4.09 (m, 1H), 3.91 (d,J=4.40 Hz, 2H), 3.84 (s, 3H), 3.76 (s, 3H), 3.62. (d,J=12.80 Hz, 1H), 3.46 (d,J=12.80 Hz, 1H), 3.01 (d,J=11.20 Hz, 2H), 2.86 (t,J=6.0 Hz, 2H), 2.63 (t,J=11.20, HZ, 1H), 2.51 (m, 1H), 2.28 (s, 3H), 2.16 (t,J=11.20 Hz, 2H), 1.65 (d,J=12.0 Hz, 2H), 1.31 (m, 3H), 0.93 (d,J=5.48 Hz, 3H), ^{—}C NMR(CDCl₃,100 MHz) δ (ppm): 154.01, 152.97, 149.49, 147.52, 131.40, 121.31, 115.55 (2C), 114.64 (2C), 113,17, 112.57, 71.16, 66.78, 66.29, 62.37, 59.53, 57.34, 55.98, 55.73, 54.43 (2C) 42.25, 34.04 (2C), 30.48, 21.80. IR (KBr, cm⁻¹): 2947, 2925, 2871, 2834, 2792, 2360, 2325, 1595, 1510, 1459, 1418, 1368, 1322, 1262, 1231, 1156, 1138, 1036, 980, 937, 878, 824, 748. HRMS (ESI): m/z calcd for C₂₇H₄₁N₂O₅ (M+H)⁺: 473.3015. found: 473.2975.

### EMBODIMENT 38

synthesis of 1-(2-chlorophenoxy)-3-((3-methoxy-4-(2-(4-methylpiperidin-1-yl)ethoxy)benzyl) (methyl)amino)propan-2-ol(CHJ04085)

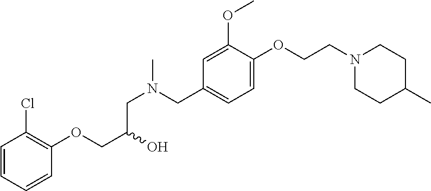 [SMILES: COc1cc(CN(C)CC(O)COc2ccccc2Cl)ccc1OCCN1CCC(C)CC1]

Compound 5 used is 1-(3-methoxy-4-(2-(4-methylpiperidin-1-yl)ethoxy)phenyl)-N-methylmethanamine (51).

Compound 7 used is 2((2-chlorophenoxy)methyl)oxirane (7-38).

Colorless oil,yield 83%, ¹H NMR (COCl₃, 400 MHz) δ (ppm): 7.34 (d,J=8.0 Hz, 1 H), 7.19 (t,J=7.60 Hz, 1H ), 6.86 (m, 5H), 4.15 (t,J=6.0 Hz, 3H), 4.03 (d,J=4.40 Hz, 2H), 3.84 (s, 3H), 3.63 (d, J=13.2 Hz, 1H ), 3.47 (d,J=13.2 Hz, 1H ), 3.00 (d, J=13.14 Hz, 2H), 2.85 (t,J=6.40 Hz, 2H), 2.70 (m, 1H ), 2.58 (m, 1H ), 2.30 (s, 3H), 2.13 (t,J=11.22 Hz, 2H), 1.64 (d,J=12.06 Hz, 2H), 1.30 (m, 3H), 0.93 (d,J=5.80 Hz, 3H). ¹³CNMR(CDCl₃,100 MHz) δ ppm): 154.31, 149.41, 147.52, 131.30, 130.25, 127.69. 123.15, 121.72, 121.34, 113.80, 112.97, 112.53, 71.46, 66.64, 66.24, 62.42, 59.34, 57.36, 55.95, 54.41 (2C), 42.40, 34.08 (2C), 30,58, 21.83. IR (KBr, cm⁻¹): 2925, 2872, 2845, 2792, 2360, 2325, 1591, 1512, 1486, 1455, 1418, 1368, 1322, 1276, 1258, 1232, 1158, 1137, 1084, 1061, 980, 937, 877, 807, 749, 693. HRMS (ESI): m/z calcd for C₂₆H₃₈ClN₂O₄ (M+H)⁺: 477.2520. found: 477.2506.

### EMBODIMENT 39

synthesis of 1-((3-methoxy-4-(2-(4-methylpiperidin-1-yl)ethoxy)benzyl)(methyl)amino-3-(2-methoxyphenoxy)propan-2-ol (CHJ04086)

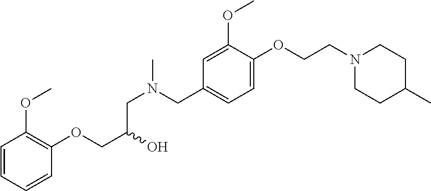 [SMILES: COc1cc(CN(C)CC(O)COc2ccccc2OC)ccc1OCCN1CCC(C)CC1]

Compound 5 used is 1-(3-methoxy-4-(2-(4-methylpiperidin-1-yl)ethoxy)phenyl)-N-methylmethanamine (51).

Compound 7 used is 2-((2-methoxyphenoxy)methyl)oxirane (7-39).

Colorless oil,yield 85%,¹H NMR (CDCl₃, 400 MHz) δ (ppm): 6.86 (m, 7H), 4.16 (t,J=6.0 Hz, 3H), 4.02 (m, 2), 3.84 (s, 6H), 3.60 (d,J'13.20 Hz, 1H), 3.47 (d, J=13.20 Hz, 1H), 3.03 (d,J=11.20 Hz, 2H), 2.87 (t,J=6.0 Hz, 2H), 2.63 (m,1H), 2.54 (m, 1H), 2.28 (s, 3H), 2.16 (t,J=10.8 Hz, 2H), 1.65 (d,J=12.0 Hz, 2H), 1.33 (m, 3H), 0.94 (d,J=5.60 Hz, 3H). ¹³CNMR(CDCl₃100 MHz)δ (ppm): 149.80, 149.37, 148.39, 147.38, 131.50, 121.78, 121.30, 120.93, 114.58, 112.97, 112.50, 112.04, 72,39, 66.46, 66.41, 62.38, 59.50, 57.31, 55.88 (2C), 54,33 (2C), 42.35, 33.89 (2C), 30.45, 21.77. IR (KBr, cm⁻¹): 2925, 2872, 2838, 2792, 2360, 2340, 1593, 1556, 1510, 1458, 1418, 1368, 1328, 1250, 1226, 1258, 1157, 1125, 1090, 1030, 980, 939, 876, 807, 744. HRMS (ESI): (ESI): m/z calcd for C₂₇H₄₁N₂O₅ (M+H)⁺: 473.2972. found: 473.2972.

### EMBODIMENT 40

synthesis of 1-(3-bromo-4-methylphenoxy)-3-((3-methoxy-4-(2-(pyrrolidin-1-yl)ethoxy)benzyl) (methyl)amino)propan-2-ol (CHJ04064)

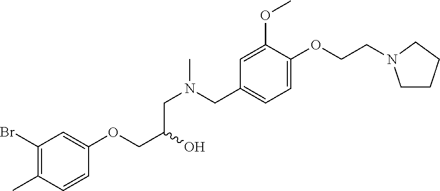 [SMILES: COc1cc(CN(C)CC(O)COc2ccc(C)c(Br)c2)ccc1OCCN1CCCC1]

Compound 5 used is 1-(3-methoxy-4-(2(pyrrolidin-1-yl)ethoxy)phenyl)-N-methylmethanamine (52).

Compound 7 used is 2-((3-bromo-4-methylphenoxy)methyl)oxirane 7-18).

Colorless oil,yield80%,¹H NMR (CD₃OD, 400 MHz) δ (ppm): 7.15 (d,J=8.40 Hz, 1H), 7.09 (s, 1H), 6.98 (s, 1H), 6.3 (m, 3H), 4.13 (t,J=5.20 Hz, 2H), 4.05 (m, 1H), 3.96 (m, 1H), 3.82 (m, 4H); 3.54 (m, 2H), 3.04 (t,J=5.20 Hz, 2H), 2.83 (s, 4H), 2.61 (dd, J=12.40, 5.6 Hz 1H ), 2.49 (dd,J=12.80, 6.80 Hz,1H ), 2.30 (d,J=6.80 Hz, 6H), 1.87 (s, 4H). ¹³CNMR (CD₃OD, 100 MHz)δ (ppm): 157.76, 149.69, 147.25, 132.08, 130.80, 129.43, 124.18, 121.47, 117.94, 113.75, 113.64, 112.98, 70.76, 67.36, 67.27, 62.21, 58.95, 54.94, 54.50, 54.23 (2C), 42.08, 22.77 (2C). 20.50, IR (KBr, cm⁻¹) 2924, 2873, 2850, 2793, 2360, 2339, 1604, 1513, 1492, 1459, 1418, 1369, 1325, 1263, 1235, 1139, 1031, 976, 928, 863, 805, 750, 669. HRMS (ESI): (ESI): m/z calcd for C₂₅H₃₆BrN₂O₄ (M+H)⁺: 507.1858. found: 507.1858.

### EMBODIMENT 41

synthesis of 1-(-bromo-5-(trifluoromethyl)phenoxy)-3-((3-methoxy-4-(2-(pyrrolidin-1-yl)ethoxy) benzyl)(methyl)amino)propan-2-ol(CHJ04065)

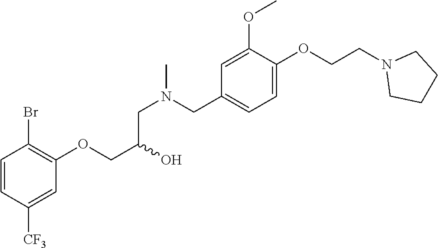 [SMILES: COc1cc(CN(C)CC(O)COc2cc(C(F)(F)F)ccc2Br)ccc1OCCN1CCCC1]

Compound 5 used is 1-(3-methoxy-4-(2-pyrrolidin-1-yl)ethoxy)phenyl)-N-methylmethanamine (52).

Compound 7 used is 2-((2-bromo-5-(trifluoromethyl)phenoxy)methyl)oxirane 7-19).

Colorless oil,yield 82%,¹H NMR (CD₃OD, 400 MHz)δ (ppm): 7.71 (d, J=8.0 Hz, 1H), 7.27 (s, 1H), 7.16 (d, J=8.0 Hz, 1H), 6.97 (s, 1H), 6.84 (m, 2H), 4.10 (m, 5H), 3.75 (s, 3H), 3.55 (s, 2H), 3.07 (t, J=5.20 Hz, 2H), 2.87 (s, 4H), 2.73 (dd, J=12.80, 4.80 Hz, 1H), 2.60 (dd, J=12.40, 6.40 Hz, 1H), 2.34 (s, 3H), 1.89 (s, 4H), 13CNMR (CD30D, 100 MHz) δ (ppm): 155.80, 149.68, 147.16, 133.70, 132,16, 130.42, 121.44, 118.14, 116.09,, 113.78, 112.88, 109.57, 71.55, 67.09, 62.34, 58.80, 54.86, 54.45, 54.22 (2C), 42.08, 23.39, 22.76 (2C), 12.54, IRl (KBr, cm⁻¹): 2968, 2938, 2879, 2793, 2361, 2323, 1734, 1700, 1518, 1492, 1459, 1419, 1398, 1328, 1268, 1252, 1167, 1137, 1080, 1035, 977, 906, 861, 748, 670. HRMS (ESI): m/z calcd for C₂₅H₃₃BrF₃N₂O₄ (M+H)⁺: 561.1576. found: 561.1569.

### EMBODIMENT 42

synthesis of 1-(3,5-dichlorophenoxy)-3-((3-methoxy-4-(2-(pyrrolidin-1-yl)ethoxy)benzyl)(methyl) amino)propan-2-ol (CHJ04066)

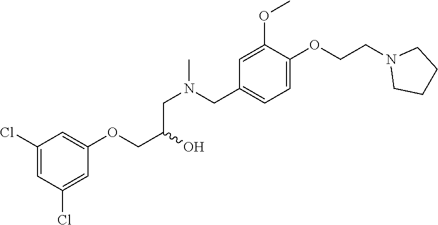 [SMILES: COc1cc(CN(C)CC(O)COc2cc(Cl)cc(Cl)c2)ccc1OCCN1CCCC1]

Compound 5 used is 1-(3-methoxy-4-(2-(pyrrolidin-1-yl)ethoxy)phenyl)-N-methylmethanamine (52).

Compound 7 used is 2-((3,5-dichlorophenoxy)methyl)oxirane 7-20).

Colorless oil,yield 84%, ¹H NMR (CD₃OD 400 MHz) δ (ppm): 6.98 (d, J=3.60 Hz, 2H), 6.87 (m, 3H), 6.82 (d, J=8.40 Hz, .1H), 4.13 (t, J=4.80 Hz, 2H), 4.03 (m, 2H), 3.88 (m, 1H), 3.79 (s, 3H), 3.52 (q, J=12.80 Hz, 2H), 3.06 (t, J=5.20 Hz, 2H), 2.85 (s, 4H), 2.62 (dd, J=12.40, 6.0 Hz, 1H), 2.47 (dd, J=12.40, 6.40 Hz, 1H), 2.32 (s, 3H), 1.88 (s, 4H). ¹³CNMR (CD₃OD, 100 MHz) δ (ppm): 160.34, 149.67, 147.23, 135.12, 132.16, 121.46, 120.37, 113.68, 113.40 (3C), 112.94, 71.00, 67.21, 67.17, 62.25, 58.63, 54.92, 54.48, 54.24 (2C), 42.20, 22.76 (2C). IR (KBr, cm−1): 2933, 2877, 2843, 2787, 2361, 2340, 1591, 1514, 1454, 1421, 1330, 1290, 1261, 1230, 1165, 1130, 1089, 1028, 964, 908, 875, 808, 752, 692, 661, 617. HRMS (ESI): m/z calcd for C₂₄H₃₃Cl₂N₂O₄ (M+H)⁺: 483.1817. found: 483.1801.

### EMBODIMENT 43

synthesis of 1-((3-methoxy-4-(2-(pyrrolidin-1-yl)ethoxy)henzyl)(methyl)amino)-3-(2,4,6-tribromophenoxy)propan-2-ol (CHJ04068)

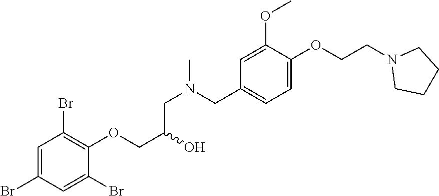 [SMILES: COc1cc(CN(C)CC(O)COc2c(Br)cc(Br)cc2Br)ccc1OCCN1CCCC1]

Compound 5 used is 1-(3-methoxy-4-(2-pyrrolidin-1-yl)ethoxy)phenyl)-N-methylmethanamine (52).

Compound 7 used is 2-((2,4,6-tribromophenoxy)methyl)oxirane 7-22).

White solid,yield 84%,mp: 60-70° C.,¹H NMR (CD₃OD, 400 MHz) δ (ppm): 7.76 (s, 2H), 6.99 (s, 1H), 6.86 (m, 2H), 4.23 (m, 1H), 4.11 (t, J=5.20 Hz, 2H), 3.97 (s, 2H), 3.79 (s, 3H), 3.54 (q, J=12.80 Hz, 2H), 2.94 (t, J=5.60 Hz, 2H), 2.72 (s. 5H), 2.55 (dd, J=12.80, 7.60 Hz, 1H ), 2.32 (s, 3H), 1.83 (s, 4H). ¹³CNMR (CD3OD, 100 MHz) δ (ppm): 152.74, 149.66, 147.42, 134.96 (2C), 134.96, 131.75, 121.53, 118.54, 117.14, 113.56, 113.07, 75.82, 67.94, 67.68, 62.12, 59.35, 55.01, 54.58, 54.23 (2C), 42.05, 22.83 (2C). IR (KBr, cm⁻¹): 3103, 2924, 2873, 2808, 1695, 1597, 1514, 1435, 1371, 1334, 1253, 1138, 1031, 989, 852, 798, 734, 684, 570, HRMS. (ESI): m/z calcd for C₂₄H₃₂Br₃N₂O₄ (M+H)⁺: 648.9912. found: 648.9938.

### EMBODIMENT 44

synthesis of 1-(3,4-dichlorophenoxy)-3-((3-methoxy-4-(2-(pyrrolidin-1-yl)ethoxy)benzyl)(methyl) amino)propan-2-ol (CHJ04072)

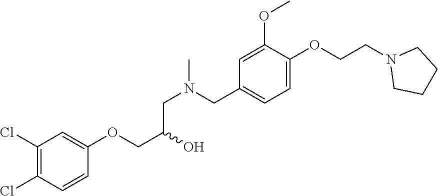 [SMILES: COc1cc(CN(C)CC(O)COc2ccc(Cl)c(Cl)c2)ccc1OCCN1CCCC1]

Compound 5 used is 1-(3-methoxy-4-(2-(pyrrolidin-1yl)ethoxy)phenyl)-N-methylmethanamine (5-2).

Compound 7 used is 2-((3,4-dichlorophenoxy)methyl)oxirane (7-26).

Colorless oil,yield 85%, ¹H NMR (CD₃OD, 400 MHz) δ (ppm): 7.37 (d, J=8.80 Hz, 1H), 7.06 (s, 1H ), 6.96 (s, 1H), 6.85 (m, 3H), 4.11 (t, J=5.20 Hz, 2H), 4.04 (m, 1H) 3.98 (m, 1H), 3.86 (m, 1H), 3.77 (s, 3H), 3.51 (q, J=12.80 Hz, 2H), 2.97 (t, j=5.20 Hz, 2H), 2.76,(s, 4H), 2.61 (dd, J=6.0 Hz, 1H), 2.47 (dd, J=12.80, 6.80 Hz, 1H), 2.32 (s, 3H), 1.85 (s, 4H). ¹³CNMR(CD₃OD, 100 MHz) δ (ppm): 158.36, 149.64, 147.37, 132.25, 131.99, 130.48, 123.32, 121.45, 116.13, 114.57, 113.50, 112.99, 70.96, 67.30, 62.27, 58.73, 54.92, 54.56, 54.24 (2C). 53.40, 42.22, 22.81 (2C), IR (KBr, cm⁻¹): 2926, 2875, 2851, 2802, 2361, 2340, 1736, 1651 , 1593, 1563, 1512, 1475, 1462, 1418, 1368, 1328, 1284, 1262, 1230, 1127, 1035, 976, 932, 902, 860, 805, 752, 671. HRMS (ESI): m/z calcd for C₂₄H₃₃Cl₂N₂O₄ (M+H)⁺: 483.1817. found: 483.1790.

### EMBODIMENT 45

synthesis of 1-(4-bromo-2,6-dichlorophonoxy)-3-((3-methoxy-4-(2-(pyrrolidin-1-yl)ethoxy)benzyl)(methyl)amino)propan-2-ol (CHJ04074)

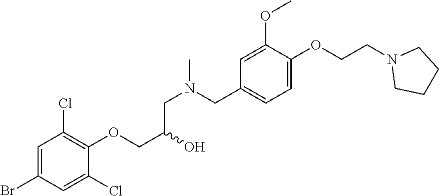 [SMILES: COc1cc(CN(C)CC(O)COc2c(Cl)cc(Br)cc2Cl)ccc1OCCN1CCCC1]

Compound 5 used is 1-(3-methoxy-4-(2-(pyrrolidin-1-yl)ethoxy)phenyl)-N-methylmethanamine (5-2),

Compound 7 used is 2-((4-bromo-2,6-dichlorophenoxy)methyl)oxirane (7-28).

Colorless oil,yield 85%, ¹H NMR (CD₃OD, 400 MHz) δ (ppm): 7.59 (s, 2H), 7.00 (s, 1H ), 6.91 (d, J=8.04 Hz, 1H), 6.85 (d, J=8.09 Hz, 1H), 4.16 (m, 3H), 4.00 (m, 2H), 3.81 (s, 3H), 3.56 (d, J=6.47 Hz, 2H), 3.09 (t, J=5.24 Hz, 2H), 2.89 (s, 4H), 2.69 (dd, J=5.54, 4.71 Hz, 1H.) 2.57 (dd, J=12.76, 7.5 Hz, 1H), 2.32 (s, 3H), 1.90 (s, 4H). ¹³CNMR (CD₃OD, 100 MHz) δ (ppm): 150.90, 149.71, 147.19, 131.98, 131.46 (3C), 129.99, 121.53, 116,16, 113.92, 113.01, 76.00, 67,88, 67.09, 62.06, 59.18, 54.95, 54.44, 54.23 (2C), 41.93, 22.75 (2C). IR (KBr, cm−1): 2953, 2920, 2866, 2765, 1726, 1651, 1593, 1516, 1458, 1419, 1373, 1327, 1261, 1230, 1136, 1085, 1028, 964, 875, 842, 800, 752, 557. HRMS (ESI): m/z calcd for C₂₄H₃₂BrCl₂N₂O₄ (M+H)⁺: 561.0923. found: 561.0882.

### EMBODIMENT 46

synthesis of 1-(3-bromo-5-chlorophenoxyl-3-((3-metboxy-4-(2-(pyrrolidin-1-yl)ethoxy)benzyl)methyl)aminopropan-2-ol (CHJ04075)

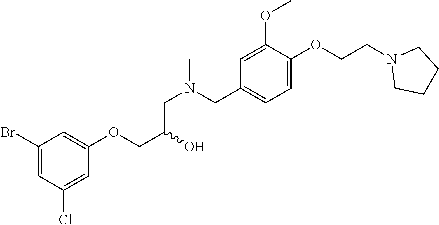 [SMILES: COc1cc(CN(C)CC(O)COc2cc(Cl)cc(Br)c2)ccc1OCCN1CCCC1]

Compound 5 used is 1-(3-methoxy-4-(2-pyrrolidin-1-yl)ethoxy)phenyl)-N-methylmethanamine (5-2).

Compound 7 used is 2-((3-bromo-5-chlorophenoxy)methyl)oxirane (7-29).

Colorless oil,yield 87%, ¹H NMR (CD₃OD, 400 MHz) δ (ppm); 7.13 (s, 1H), 7.03 (s, 1H), 6.98 (s, 1H), 6.90 (m, 2H), 6.82 (d, J=8.07 Hz, 1H), 4.15 (t, J=5.54 Hz, 2H), 4.03 (m, 2H), 3.88 (m, 1H), 3.80 (s, 3H), 3.52 (q, J=12.86 Hz, 2H), 3.12 (t, J=5.24 Hz, 2H), 2.92 (s, 4H), 2.62 (dd, J=12.72, 6.12 Hz, 1H), 2.48 (dd, J=12.71, 6.44 Hz, 1H), 2.33 (s, 3H), 1.91 (s, 4H), ¹³CNMR (CD₃₆l OD, 100 MHz) δ (ppm): 160.38, 149.69, 147.13, 135.27, 132.25, 123.18, 122.49, 121.48, 116.32, 113.84 (2C), 112.93 70.99, 67.18, 62.23, 58.61, 54.94, 54.42, 54.25 (2C), 42.19, 22.74 (2C), 12.54.IR (KBr, cm⁻¹): 2974, 2934, 2379, 2309, 1716, 1651, 1593, 1557, 1539, 1510, 1475, 1419, 1393, 1339, 1231, 1124, 1038, 854, 670, 520. HRMS (ESI): m/z calcd for C₂₄H₃₃BrClN₂O₄ (M+H)⁺: 527.1312. found: 527.1275.

### EMBODIMENT 47

synthesis of 1-((4-(2-(diethylamino)ethoxy)-3-methoxybenzyl)(methyl)amino)-3-(2-isopropylphenoxy)propan-2-ol (CHJ04089)

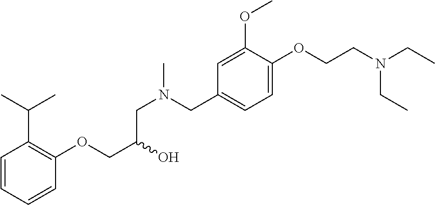 [SMILES: CCN(CC)CCOc1ccc(CN(C)CC(O)COc2ccccc2C(C)C)cc1OC]

Compound 5 used is N,N-diethyl-2-(2-methoxy-4-((methylamino)methyl)phenoxy)ethan-1-amine 5-3),

Compound 7 used is 2-((2-isopropylphenoxy)methyl)oxirane (7-7).

Colorless oil,yield 82%, ¹H NMR (CDCl₃, 400 MHz) δ (ppm): 7.20 (d, J=7.60 Hz, 1H), 7.13 (t, J=8.0 Hz, 1H), 6.93 (t, J=07.20 Hz, 1H), 6.82 (m, 4H), 4.11 (m, 3H), 3.98 (m, 2H), 3.84 (s, 3H), 3.65 (d, J=12.80 Hz, 1H), 3.46 (d, J=12.80 Hz, 1H), 3.26 (m, 1H), 2.94 (t, J=6.80 Hz, 2 H), 2.66 (m, 5H), 2.54 (m, 1H), 2.31 (s, 3H), 1.20 (d, J=7.20 Hz, 6H), 1.08 (t, J=7.20 Hz, 6H). ¹³CNMR(CDCl₃,100 MHz) δ (ppm): 155.82, 149.38, 147.59, 137.05, 131.20, 126.55, 126.07, 121.26, 120.89, 112.69, 112.41, 111.30, 70.35, 67.24, 66.34, 62.47, 59.66, 55.94, 51.66, 47.85 (2C), 42.36, 26.89, 22.63 (2C), 11.78 (2C). IR (KBr, cm−1): 3035, 2965, 2933, 2871, 2360, 1598, 1513, 1491, 1451, 1417, 1368, 1286, 1261, 1239, 1197, 1139, 1088, 1034, 983, 938, 881, 823, 751. HRMS (ESI): m/z calcd for C₂₇H₄₃N₂O₄ (M+H)⁺: 459.3223. found: 459.3166.

### EMBODIMENT 48

synthesis of 1-(2-bromo-5-(trifluoromethyl)phenoxy)-3-((4-(2-(diethylamino)ethoxy)-3-methoxy benzyl)(methyl)amino)propan-2-ol (CHJ04090)

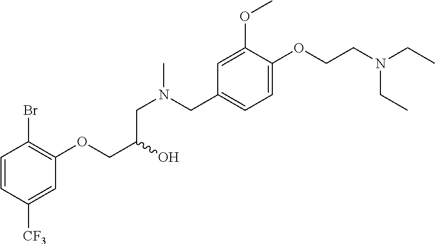 [SMILES: CCN(CC)CCOc1ccc(CN(C)CC(O)COc2cc(C(F)(F)F)ccc2Br)cc1OC]

Compound 5 used is N,N-diethyl-2-(2-methoxy-4-((methylamino)methyl)phenoxy)ethan1-amine (5-3).

Compound 7 used is 2-((2bromo-5-(trifluoromethl)phenoxy)methyl)oxirane (7-19).

Colorless oil,yield 81%, ¹H NMR (CDCl₃, 400 MHz) δ (ppm): 7.63 (d, J=8.40 Hz, 1H), 7.10 (m, 2H), 6.81 (m, 3H), 4.11 (m, 5H), 3.84 (s, 3H), 3.65 (d, J=12.80 Hz, 1H), 3.47 (d, J=13.20 Hz, 1H), 2.94 (t, J=6.80 Hz, 2H), 2.67 (m, 5H), 2.55 (m, 1H.) 2.32 (s, 3H), 1.08 (t, J=7.20 Hz, 6H.). ¹³CNMR (CDCl₃, 100 MHz) δ (ppm): 155.44, 149.39, 147.63, 133.70 (2C), 131.09, 121.31 (2C), 118.73, 116.43, 112.73, 112.45, 109.89, 71.63, 67.28, 66.10, 62.48, 58.95, 55.94, 51.65, 47.85 (2C), 42.42, 11.77 (2C). IR (KBr, cm⁻¹): 2969, 2936, 2876, 2837, 2360, 2340, 1651, 1597, 1539, 1512, 1488, 1461, 1420, 1329, 1296, 1263, 1231, 1169, 1128, 1081, 1036, 979, 934, 857, 814, 751, 656, 565. HRMS (ESI): m/z calcd for C₂₅H₃₅BrF₃N₂O₄ (M+H)⁺: 563.1676. found: 563.1676.

### EMBODIMENT 49

synthesis of 1-(3,4-dichlorophenoxy)-3-((4-(2-(diethylamino)ethoxy)-3-methoxybenzyl)(methyl) amino)propan-2-ol (CHJ04091)

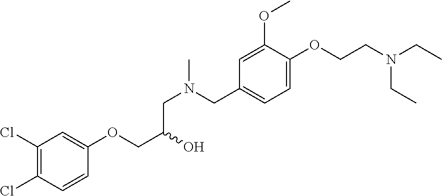 [SMILES: CCN(CC)CCOc1ccc(CN(C)CC(O)COc2ccc(Cl)c(Cl)c2)cc1OC]

Compound 5 used is N,N-diethyl-2-(2-methoxy-4-((methylamino)methyl)phenoxy)ethan-1-amine (5-3),

Compound 7 used is 2-((3,4-dichlorophenoxy)methyl)oxirane (7-26).

Colorless oil,yield 85%, ¹H NMR (CDCl₃, 400 MHz) δ (ppm): 7.31 (d, J=8.8 Hz, 1H), 7.00 (s, 1H), 6.80 (m, 4H), 4.09 (m, 3H), 3.91 (m, 2H), 3.85 (s, 3H), 3.62 (d, J=12.80 Hz, 1H), 3.45 (d, J=12.80 Hz, 1H), 2.95 (t, J=6.40 Hz, 2H), 2.67 (m, 5H), 2.47 (m, 1H), 2.29 (s, 3H), 1.08 (t, J=7.20 Hz, 6H). ¹³CNMR(CDCl₃,100 MHz) δ (ppm): 157.83, 149.37, 147.63, 132.81, 131.03, 130.65, 124.20, 121.31, 116.45, 114.63, 112.73, 112.46, 70.97, 67.20, 65.97, 62.37, 59.08, 55.96, 51.65, 47.80 (2C), 42.28, 11.70 (2C). IR (KBr, cm−1): 2970, 2936, 2874, 2832, 2360, 2326, 1700, 1651, 1593, 1559, 1539, 1511, 1475, 1459, 1418, 1337, 1285, 1263, 1230, 1158, 1126, 1090, 1126, 1035, 930, 865, 806, 671. HRMS (ESI): m/z calcd for C₂₄H₃₅Cl₂N₂O₄ (M+H)⁺: 485.1974. found: 485.1904.

### EMBODIMENT 50

synthesis of 1-(3-bromo-4-fluorophenoxy)-3-((4-(2-(diethylamino)ethoxy)-3-methoxybenzyl)(methyl)amino)propan-2-ol (CHJ04092)

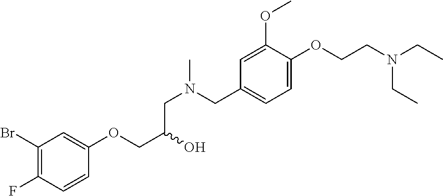 [SMILES: CCN(CC)CCOc1ccc(CN(C)CC(O)COc2ccc(F)c(Br)c2)cc1OC]

Compound 5 used is N,N-diethyl-2-(2-methoxy-4-((methylamino)methyl)phenoxy)ethan-1-amine (5-3).

Compound 7 used is 2-((3-bromo-4-fluorophenoxy)metyl)oxirane (7-21).

Colorless oil,yield 82%, ¹H NMR (CDCl₃, 400 MHz) δ (ppm): 7.09 (m, 1H), 7.02 (t, J=8.80 Hz, 1H), 6.81 (m, 4H), 4.09 (m, 3H), 3.90 (m, 2H), 3.85 (s, 3H), 3.62 (d, J=12.80 Hz, 1H), 3.46 (d, J=12.80 Hz, 1H ), 2.95 (t, J=7.20 Hz, 2H), 2.67 (m, 5H), 2.47 (m, 1H), 2.29 (s, 3H), 1.09 (t, J=7.20 Hz, 6H). ¹³CNMR(CDCk₃, 100 MHz) δ (ppm): 155.25, 149.38, 147.62, 131.06, 121.3 (2C), 119.02 (2C), 116.52, 114.98, 112.58, 109.01, 71.29, 67.19, 66.04 62.37, 59.14, 55.96, 51.64, 47.80 (2C), 42.27, 11.69 (2C). IR (KBr, cm₋₁): 2969, 2936, 2875, 2832, 2360, 2340, 1651, 1592 1556, 1539, 1511, 1493, 1459, 1418, 1373, 1333, 1262, 1220, 1203, 1158, 1139, 1092, 1036, 929, 864, 805, 752. HRMS (ESI): m/z calcd for C₂₄H₃₅BfFN₂O₄ (M+H)⁺: 513.1764. found: 513.1710.

### EMBODIMENT 51

synthesis of 1-(4-bromo-3trifluoromethyl)phenoxy)-3-(( 4-(2-(diethylamino)ethoxy)-3-methoxybenzyl)(methyl)amino)propan-2-ol (CHJ04093)

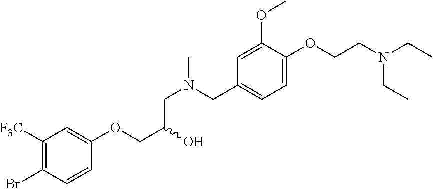 [SMILES: CCN(CC)CCOc1ccc(CN(C)CC(O)COc2ccc(Br)c(C(F)(F)F)c2)cc1OC]

Compound 5 used is N,N-diethyl-2-(2-methoxy-4-((methylamino)methyl)phenoxy)ethan-1-amine (5-3).

Compound 7 used is 2((4-bromo-3-(trifluoromethyl)phenoxy)methyl)oxirane (7-3).

Colorless oil,yield 83%, ¹H NMR (CDCl₃400 MHz) δ (ppm): 7.57 (d, J=8.80 Hz, 1H), 7.24 (s, 1H ), 6.92 (d, J=8.80 Hz, 1H ), 6.82 (m, 3H), 4.11 (t, J=6.40 Hz, 3H), 3.96 (m, 2H) 3.84 (s, 3H), 3.63 (d, J=12.80 Hz, 1H ), 3.46 (d, J=12.80 Hz, 1H), 2.97 (t, J=6.4 Hz, 2H), 2.67(m, 5H), 2.48 (m, 1H), 2.30 (s, 3H), 1.10 (t, J=7.20 Hz, 6H), ¹³CNMR(CDCl₃, 100 MHz) δ (ppm): 157.82 149.40, 147.62, 135.72 (2C), 131.02, 121.32 (2C), 118.88 (2C), 114.64, 112.77, 110.18, 71.29, 67.19, 66.04, 62.37, 59.14, 55.96, 51.64, 47.80 (2C), 42.27, 11.69 (2C). IR (KBr, cm⁻¹): 2969, 2936, 2875, 2800, 2360, 2340, 1603, 1512, 1475, 1462, 1419, 1373, 1330, 1313, 1260, 1233, 1171, 1139, 1097, 1036, 1017, 980, 932, 880, 810, 752, 702. HRMS (ESI): m/z calcd for C₂₅H₃₅BrF₃N₂O₄ (M+H)⁺: 563.1732. found: 563.1695.

### EMBODIMENT 52

synthesis of1-(4-bromo-2,6-dichlorophenoxy)-3-((4-(2-(diethylamino)ethoxy)-3-methoxybenzyl) (methyl)amino)propan-2-ol (CHJ04094)

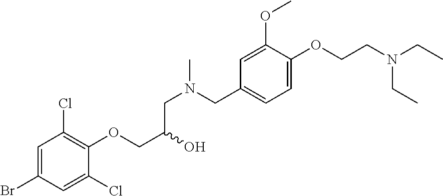 [SMILES: CCN(CC)CCOc1ccc(CN(C)CC(O)COc2c(Cl)cc(Br)cc2Cl)cc1OC]

Compound 5 used is N,N-diethyl-2-(2-methoxy-4-((methylamino)methyl)phenoxy)ethan-1-amine (5-3).

Compound 7 used is 2-((4-bromo-2,6-dichlorophenoxy)methyl)oxirane (7-28).

Colorless oil,yield 87%, ¹H NMR (CDCl₃, 400 MHz) δ (ppm): 7.44 (s, 2H), 6.81 (m, 3H), 4.06 (m, 5H), 3.84 (s, 3H), 3.61 (d, J=12.80 Hz, 1H), 3.49 (d, J−12.80 Hz, 1H), 2.96 (t, J=6.80 Hz, 2H), 2.69 (m, 5H), 2.59 (m, 1H), 2.28 (s, 3H), 1.09 (t, J=7.20 Hz, 6H). ¹³CNMR(CDCl₃, 100 MHz) δ (ppm): 150.74, 149.36, 147.52, 131.62 (3C), 131.24, 130.16, 121.30, 116.51, 112.76, 112.45, 71.29, 67.19, 66.04, 62.37, 59.14, 55.96, 51.64, 47.80 (2C), 42.27, 11.69 (2C). IR, (KBr, cm⁻¹): 2966, 2935, 2875, 2800, 2362, 2340, 1597, 1548, 1514, 1456, 1375, 1328, 1261, 1134, 1031, 995, 929, 854, 802, 748, 702, 569. HRMS (ESI): m/z calcd for C₂₄H₃₄BrCl₂N₂O₄ (M+H)⁺: 563.1079. found: 563.1039.

### EMBODIMENT 53

synthesis of 1-((4-(2-(1H-imidazol-1-yl)ethoxy)-3-methoxybenzyl)(methyl)amino)-3-(4-bromo-3-(trifluoromethyl)phenoxy)propari-ol (CHJ04097)

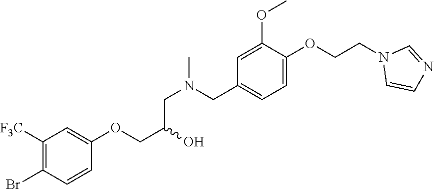 [SMILES: COc1cc(CN(C)CC(O)COc2ccc(Br)c(C(F)(F)F)c2)ccc1OCCn1ccnc1]

Compound 5 used is 1-(4-(2-(1H-imidazol-1-yl)ethoxy)-3-methoxyphenyl)-N-methylmethanamine (5-4).

Compound 7 used is 2-((4-bromo-3-(trifluoromethyl)phenoxy)methyl)oxirane. (7-3).

White solid,yield 85%, ¹H NMR (CDCl₃, 400 MHz) δ (ppm): 7.64 (s, 1H), 7.56(d, J=8.80 Hz, 1H), 7.23(s1H), 7.10(s, 1H),7.05(s, 1H),6.92(d,J=8.8 Hz, 1H),6.84(s, 1H),6.74 (m,2H) 4.34 (m,2H), 4.24(m, 2H), 4.10(m 1H), 3.96(m, 2H), 3.84 (s, 3H), 3.62 (d, J=12.80 Hz, 1H), 3.46 (d,J=12.80 Hz, 1H), 2.62 (t, J=11.20 Hz, 1H), 2.49 (m, 1H), 2.30 (s, 3H). ¹³CNMR(CDCl₃,100 MHz) δ (ppm): 157.80, 149.88, 146.88, 137.64, 135.73, 132.45, 130.69, 129.26, 121.25, 119.59, 118.87, 114.63, 114.58, 114.18, 112.82, 110.21, 70.88, 68.92, 66.00, 62.35, 59.07, 55.95, 46.69, 42.35. IR (KB, cm⁻¹): 3111, 2929, 2877, 2787, 2362, 2340, 1600, 1514, 1473, 1419, 1321, 1261, 1230, 1170, 1138, 1093, 1026, 962, 908, 871, 810, 759, 663. HRMs (ESI): m/z calcd for C₂₄H₂₈BrF₃N₃O₄ (M+H)⁺: 558.1215. found: 558.1172.

### EMBODIMENT 54

synthesis of 1-((4-(2-(1H-imidazol-1yl)ethoxy)-3-methoxybenzyl)(methyl)amino)-3-(2-bromo-5-(trifluoromethyl)phenoxy)propan-2-ol (CHJ04099)

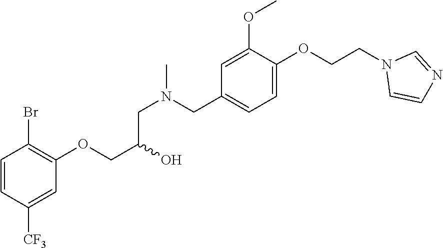 [SMILES: COc1cc(CN(C)CC(O)COc2cc(C(F)(F)F)ccc2Br)ccc1OCCn1ccnc1]

Compound 5 used is 1-(4-(2-(1H-imidazol-1-yl)ethoxv)-3-methoxyphenyl)-N-methylmethanamine (5-4),

Compound 7 used is 2-((2-bromo-5-(trifluormethyl)phenoxy)methyl)oxirane (7-19)

White solid,yield 90%, ¹H NMR (CDCl³, 400 MHz) δ (ppm): 7.62(d, J=12.40 Hz, 2H),7.08(m, 4H), 6.85(s,1H),6.78(d, J=8.0 Hz, 1H),6.71(d, J=8.0 Hz, 1H), 4.34 (m,2H), 4.23(m, 2H),4,09 (m, 3H),3.83 (s,3H),3.64 (d, J=12.80 Hz, 1H), 3.47 (d. J=13.20 Hz, 1H), 2.73 (t,J=11.60 Hz, 1H), 2.55 (m, 1H), 2.32 (s, 3H). ¹³CNMR(CDCl₃,100 MHz) δ (ppm): 155.41, 149.90, 146.85, 137.67, 133.71, 132.57, 130.71, 129.39, 122.28, 121.25, 119.58, 118.81, 116.41, 114.19, 112.80, 109.89, 71.55, 68.94, 66.15, 62.45, 58.93, 55.94, 46.65, 42.52. IR (KBr, cm⁻¹): 3118, 2879, 2845, 2787, 2362, 2340, 1676, 1595, 1516, 1460, 1421, 1388, 1332, 1290, 1259, 1138, 1114, 1085, 1029, 964, 906, 819, 752. HRMS (ESI): m/z calcd for C₂₄H₂₈BrF₃N₃O₄ (M+H)⁺: 558.1215. found: 558.1160.

### EMBODIMENT 55

synthesis of 1-((4-(2-(1H-imidazol-1-yl)ethoxy)-3-methoxybenzyl)(methyl)amino-3-(4-bromophenoxy)propan-2-ol (CHJ05001)

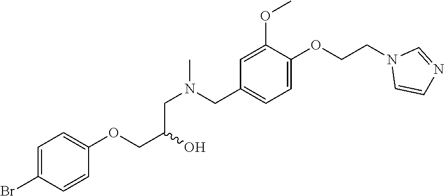 [SMILES: COc1cc(CN(C)CC(O)COc2ccc(Br)cc2)ccc1OCCn1ccnc1]

Compound 5 used is 1-(4-(2-(1H-imidazol-1-yl)ethoxy)-3-methoxyphenyl)-N-methylmethanamine (5-4).

Compound 7 used is 2-((4-bromophenoxy)methyl)oxirane (7-12).

White solid,yield 85%, ¹H NMR (CDCl₃, 400 MHz) δ (ppM): 7.63(s, 1H)7.35(d, J=8.40 Hz, 2H), 7.08(d, J=14.80 Hz, 2H), 6.78(m, 5H),4.34 (m,2H), 4.23(m, 2H),4.09 (m, 1H),3.92 (d, J=4.40 Hz, 2H), 3.83 (s, 3H), 3.61 (d, J=13.20 Hz, 1H), 3.45 (d, J=12.80 Hz, 1H), 2.62 (t, J=11.60 Hz, 1H), 2.49 (m, 1H), 2.29 (s, 3H). ¹³CNMR(CDCl₃, 100 MHz) δ (ppm): 157.89, 149.88, 146.84, 137.67, 132.56, 132.24 (2C), 129.38, 121.23, 119.58, 116.36 (2C), 114.19, 113.12, 112.81, 70.60, 68.95, 66.12, 62.34, 59.31, 55.96, 46.65, 42,34. IR (KBr, cm⁻¹): 3111, 2931, 2879, 2843, 2771, 2362, 2340, 1712, 1587, 1514, 1487, 1456, 1419, 1355, 1325, 1242, 1139, 1099, 1064, 1028, 999, 960, 910, 883, 858, 819, 754, 692, 663, 615. HRMS (ESI): m/z calcd for C₂₃H₂₉BrN₃O₄ (M+H)⁺: 490.1341. found: 490.1341.

### EMBODIMENT56

synthesis of 1-((4-(2-(1H-imidazol-1-yl)ethoxy)-3-methoxybenzyl)(methyl)amino)-3-(3-bromo-4-methylphenoxy)propan-2-ol (CHJ05002)

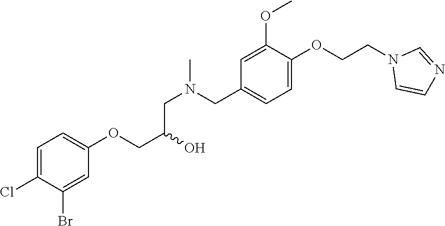 [SMILES: COc1cc(CN(C)CC(O)COc2ccc(Cl)c(Br)c2)ccc1OCCn1ccnc1]

Compound 5 used is 1-(4-(2-(1H-imidazol-1-yl)ethoxy)-3-methoxyphenyl)-N-methylmethanamine (5-4).

Compound 7 used is 2-((3-bromo-4-chlorophenoxy)methyl)oxirane (7-17).

White solid,yield 90%, ¹H NMR (CDCl₃, 400 MHz) δ (ppm): 7.63(s, 1H),7.30(d, J=8.80 Hz, 1H), 7.16(s, 1H), 7.08(d, J=16.0 Hz, 2H), 6.78(m, 4H)4.34 (m,2H), 4.23(m 2H)4.09 (m, 1H),3.92 (m, 2H), 3.84 (s, 3H), 3.61 (d, J=13.2 Hz,1H),3.45 (d, J=13.2 Hz, 1H), 2.61 (t, J=11.60 Hz, 1H), 2.48 (m, 1H), 2.29 (s,3H). ¹³CNMR(CDCl₃, 100 MHz) δ (ppm): 157.73, 1.49.89, 146.87, 137.67, 132.50, 130.48, 129.37, 126.16, 122.52, 121.24, 119.55 (2C), 115.30, 114.19, 112.81, 70.74, 68.95, 66.04, 62.35, 59.12, 55.97, 46.66, 42.37. IR (KBr, cm−1): 3113, 2927, 2877, 2845, 2785, 2362, 2340, 1589, 1564, 1512, 1467, 1419, 1384, 1323, 1261, 1239, 1141, 1089, 1029, 960, 906, 858, 808, 754, 666. HRMS (ESI): m/z calcd for C₂₃H₂₈BrClN₃O₄ (M+H)⁺: 524.0952. found: 524.0916.

### EMBODIMENT 57

synthesis of 1-((4-(2-(1H-imidazol-1-yl)ethoxy)03-methoxybenzyl)(methyl)amino)-3-(3-bromo-4-methylphenoxy)propan-2-ol (CHJ05003)

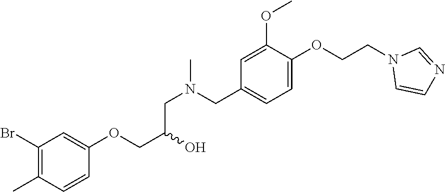 [SMILES: COc1cc(CN(C)CC(O)COc2ccc(C)c(Br)c2)ccc1OCCn1ccnc1]

Compound 5 used is 1-(4-(2-(1H-imidazol-1-yl)ethoxy)-3-methoxyphenyl)-N-methylmethanamine (5-4).

Compound 7 used is 2((3-bromo-4-methylphenoxy)methyl)oxirane (7-18).

White solid,yield 90%,¹H NMR CDCl₃, 400 MHz) δ (ppm): 7.64(s, 1H),7.10(s, 3H), 7.06(s, 1H),6.86(s, 1H ), 6.73(m, 3H),4.34 (m, 2H), 4.24(m, 2H),4.09 (m, 1H),3.92 (m, 2H), 3.84 S,3H),3.61 (d, J=12.80 Hz, 1H),3.46 (d, J=13.20 Hz, 1H ), 2.62 (t, J=12.0 Hz, 1H), 2.48 (m, 1H ), 2.30 (d,J=10.40 Hz, 6H), ¹³CNMR(CDCl₃,100 MHz) δ (ppm): 157.34, 149.86, 146.83 137.67, 132.49, 130.99, 130.04, 129.37, 124.80, 121.24, 119.60, 118.30. 114.15, 113.90, 112.77, 70.68, 68.93, 66.11, 62.33, 59.30, 55.96, 46.64, 42.30, 21.84. IR (KBr, cm−1): 3112, 2924, 2877, 2844, 2777, 2361, 2340, 1605, 1564, 1511, 1492, 1454, 1418, 1264, 1239, 1226, 1158, 1141, 1091, 1029, 962, 896, 866, 815, 800, 764, 739, 658, 614. HRMS (ESI): m/z calcd for C₂₄H₃₁BrN₃O₄ (M+H)⁺: 504.1498. found: 504.1460.

### EMBODIMENT 58

synthesis of 1-((4-(2-(1H-imidazol-1-yl)ethoxy)-3-methoxybenzyl)(methyl)amino)-3-(3-5-chlorophenoxy)propan-2-ol (CHJ05004)

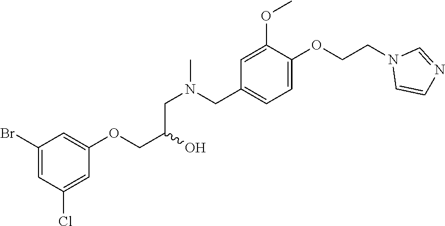 [SMILES: COc1cc(CN(C)CC(O)COc2cc(Cl)cc(Br)c2)ccc1OCCn1ccnc1]

Compound 5 used is 1-(4-(2-(1H-imidazol-1-yl)ethoxy)-3-methoxyphenyl)-N-methylmethanamine (5-4).

Compound 7 used is 2-((3-bromo-5-chlorophenoxy)methyl)oxirane (7-29),

White solid,yield 88%, ¹H NMR (CDCl₃, 400 MHz) δ (ppm): 7.63(s, 1H.), 7.10(s, 2H), 7.06(s, 1H ), 6.96(s, 1H )6.84(s, 2H),6.73(m, 2H),4.35 (m,2H), 4.24(m, 2H)4.07 (m, 1H )3.93 (m, 2H), 3.84 (s,3H), 3.61 (d, J=12.94 Hz,1H ),3.45 (d, J=13.35 Hz, 1H ), 2.60 (t, J=11.55 Hz, 1H), 2.47 (m, 1H), 2.29 (s, 3H). ¹³CNMR(CDCl₃, 100 MHz) δ (ppm): 159.85, 149.91, 146.89, 137.67, 135.53, 132.46, 129.39 124.03, 122.83, 121.24, 119.58, 116.57, 114.21 (2C), 112.79, 70.90, 68.95, 65.96, 62.35, 59.05, 55.97, 46.66, 42.37. HRMS (ESI): m/z calcd for C₂₃H₂₈BrClN₃O₄ (M+H)⁺:524.0952. found: 524.0913.

### Experiment on Compounds in the Invention in Inhibiting the Activity of Tumor Cell Growth

### 1 Experiment Materials

Dissolve CHJ compound series with dimethyl sultbxide (DMSO, final concentration 0.4%), and use RPMI-1640 culture medium containing 15% fetal calf serum to prepare 1 mg/mL solution for future use. During drug administration in groups, use the culture medium to dilute it to the concentration needed.

Experiment reagent

| Reagent name | Specification | | Manufacturer |
|---|---|---|---|
| Cis-platinum | 10 | mg/bottle | Qilu Pharmaceutical Co., |
| | | | Ltd. |
| DMEM (High | 500 | mL | Biological Industries in |
| Glucose) | | | Isreal |
| Fetal calf serum | 500 | mL | Biological Industries in |
| | | | Isreal |
| Dimethyl sulfoxide | 50 | mL | Beijing Solarbio Life |
| | | | Sciences Co., Ltd.: |
| MTT | 5 | mL | Sigma in America |
| EDTA pancreatic | 100 | mL | Beijing Solarbio Life |
| enzyme | | | Sciences Co., Ltd.: |

Cell lines: human lung cancer (A549), Inman ovarian cancer (SKOV3), human melanoma (A375) and human colon cancer (LOVO) cell lines, all purchased from the Cell Bank of the Chinese Academy of Sciences, DMEM containing 15% fetal bovine serum (High Glucose) culture medium, placed in a 37° C., 5% CO2 incubator.

### 2. Experimental Methods

MTT is an oxidizing yellow dye with the chemical name of 3-(4,5-dimethyl-2-thiazolyl)-2,5-diphenyl-2-H-tetrazoliumbromide tetrazole bromide. The MMT method, also known as the MTT colorimetric method, can test the survival and growth of cells. The method is simple and easily accessible and often used to screet the substance with cytotoxic activity. The basic principle of testing, is that succinodehydrogenase can reduce exogenous MIT so that MTT is reduced to blueviolet crystal Formazan insoluble to water. The crystal deposits in cells. The succinodehydrogenase exists in the mitochondria of living cells, not in the dead cells. Therefore, the chromogenic reaction can only take place in living cells. Then, dissolve the blueviolet crystal Formazan contained in cells with dimethyl sulfoxide (DMSO), and measure its absorbancy with microplate reader so that the count of living cells is reflected indirectly. Within the scope of certain cell count, the amount of blueviolet crystal Formazan formed is positively correlated to the count of living cells.

MTT is efficient, accurate, convenient, economical and highly repeatable. It has been extensively applied to the screening of antitumor drugs, activity detection of medical bioactive factor, cytotoxic activity test, and the measurement of tumor radiosensitivity.

Preparation of 5 mg/mL MTT solution: weigh 500.0 mg MTT powder and dissolve it in 100 ml warm PBS. Use the millipore filter of 0.22 μm aperture to screen the bacteria and obtain the filtrate. Divide the filtrate into small doses and put them the centrifugal tube which has gone through autoclaved sterilization. Freeze them in a dark place under −20° C.

Fetch the frozen tube containing the tumor cells from ligrt d. nitrogen and put it swiftly into the 37° C. incubator. Keep shaking till it melts. Rub the rim of the cover of frozen tube with 75% alcohol, suck in the cell suspension and transfer it to 10 mL centrifugal tube. Add 5 ml culture medium. Apply low-speed centrifugation (25° C. 3000r/min, 5 min), discard the supernatant, add culture medium, and then repeat the centrifugation once more. After proper amount of culture medium is added for the purpose of dilution, blow away the cells with straw to form suspension. Transfer the suspension to the culture bottle, and place the bottle in the 37° C. cell incubator containing 5% CO₂. Change the culture solution the next day and place the bottle in the 37° C. cell incubator containing 5% CO² for continued culture.

Human lung cancer (A549), human ovarian cancer (SKOV3), human melanoma (A375) and human colon cancer (LOVO) ceilsare all adherent. Based on the growth rate of tumor cells, wash the adherent tumor cells in the logarithmic phase and digest them in 0.25% EDTA pancreatic enzyme. Adjust the cell count to 1×105/mL and inoculate the cells in 96-pore plate, each pore containing 100 μL. Culture the cells in the 37° C. CO₂ incubator, and administer the drug 24 h later. Add pharmaceutical (CJH compound series) of different concentrations to the administration group. Set up 5 dosage groups for each pharmaceutical, respectively 100, 10, 1, 0.1, 0.01 μmol/L. Set up three complex pores for each concentration. Set up blank control, DMSO (0.8%) solvent control and cis-platinum positive control. After 48 h of culture in the 37 ° C. incubator containing 5% CO₂, measure the OD value through MTT method and calculate the cell inhibition ratio.

### 2.3 Calculation of IC₅₀

After 48 h of culture of human lung cancer (A549), human ovarian cancer (SKOV3). human melanoma (A375) and human colon cancer (LOVO) cells, stop it and add 10 μL 0.5% MTT solution to each pore. Place it in the CO₂ incubator for 4 h. Remove the liquid from each pore, respectively add 0.2 mL DMSO solution, and oscillate it adequately on the table concentrator under low frequency so that the blueviolet crystal Formazan is dissolved adequately. Place it in the microplate reader and record the OD value at the wavelength of 490 mm. Calculate the average OD value of three parallel pores of different concentrations, and calculate the cell inhibition ratio and IC₅₀ of each tested pharmaceutical under different concentrations based on the average.

Inhibition ratio (%)=[1-tested sample OD/negative control group OD]×100%.

IC₅₀ value of target compound against cancer cell proliferation

| | IC₅₀ (μM) | | | |
|---|---|---|---|---|
| Compound | A549 | SKOV3 | A375 | LOVO |
| SAMS10 | 14.64 ± 0.06 | 21.07 ± 0.18 | 27.11 ± 0.66 | 8.48 ± 0.07 |
| CHJ02029 | 3.55 ± 0.06 | 6.30 ± 0.16 | 2.43 ± 0.12 | 1.87 ± 0.02 |
| CHJ02049 | 0.52 ± 0.23 | 0.62 ± 0.08 | 0.62 ± 0.04 | 1.25 ± 0.03 |
| CHJ02050 | 1.09 ± 1.55 | 4.04 ± 0.06 | 1.87 ± 0.07 | 3.88 ± 1.80 |
| CHJ03001 | 1.30 ± 1.03 | 2.48 ± 10.63 | 1.95 ± 0.33 | 1.26 ± 0.53 |
| CHJ03003 | 6.08 ± 0.90 | 3.00 ± 0.99 | 5.95 ± 0.42 | 0.70 ± 0.26 |
| CHJ03004 | 2.10 ± 1.25 | 6.19 ± 0.25 | 0.72 ± 0.80 | 2.54 ± 0.10 |
| CHJ03005 | 15.58 ± 0.16 | 3.99 ± 0.21 | 5.60 ± 0.81 | 6.66 ± 0.11 |
| CHJ03011 | 10.56 ± 0.02 | 21.21 ± 1.08 | 1.82 ± 1.23 | 15.19 ± 1.78 |
| CHJ03012 | 2.19 ± 1.04 | 3.54 ± 1.02 | 0.16 ± 0.33 | 1.95 ± 0.43 |
| CHJ03014 | 1.09 ± 0.06 | 6.19 ± 0.62 | 1.63 ± 0,72 | 0.53 ± 0.15 |
| CHJ03015 | 1.03 ± 0.13 | 4.47 ± 1.62 | 3.22 ± 0,42 | 0.55 ± 0.21 |
| CHJ03017 | 17.25 ± 0.79 | 6.21 ± 1.04 | 8.68 ± 0.11 | 2.44 ± 0.03 |
| CHJ03018 | 18.76 ± 1.06 | 1.79 ± 0.23 | 3.13 ± 0.06 | 1.72 ± 1.58 |
| CHJ03043 | 2.50 ± 1.32 | 2.90 ± 0.19 | 0.54 ± 0.26 | 0.95 ± 0.41 |
| CHJ04010 | 4.51 ± 1,29 | 4.96 ± 1.26 | 0.45 ± 0.46 | 2.78 ± 0.23 |
| CHJ04011 | 4.05 ± 1.12 | 2.41 ± 1.03 | 0.48 ± 0.28 | 1.01 ± 0.08 |
| CHJ04012 | 6.38 ± 0.07 | 4.34 ± 0.32 | 1.24 ± 1.01 | 2.12 ± 0.04 |
| CHJ04020 | 1.38 ± 1.14 | 3.40 ± 1.17 | 1.60 ± 0.92 | 3.83 ± 1.80 |
| CHJ04022 | 1.88 ± 0.19 | 1.03 ± 0.24 | 0.05 ± 0.01 | 0.69 ± 0.03 |
| CHJ04023 | 5.44 ± 2.54 | 8.03 ± 2.77 | 0.81 ± 0.33 | 3.07 ± 1.62 |
| CHJ04024 | 5.45 ± 1.03 | 1.37 ± 0.34 | 0.98 ± 0.76 | 18.61 ± 3.22 |
| CHJ04025 | 4.21 ± 2.24 | 3.32 ± 1.02 | 2.34 ± 1.63 | 2.55 ± 1.65 |
| CHJ04026 | 0.97 ± 0.21 | 7.45 ± 2.26 | 1.50 ± 0.06 | 0.82 ± 0.42 |
| CHJ04027 | 6.22 ± 1.34 | 3.02 ± 1.76 | 0.73 ± 0.13 | 2.35 ± 1.04 |
| CHJ04033 | 0.95 ± 0.37 | 6.22 ± 3.05 | 0.92 ± 0.07 | 2.23 ± 1.26 |
| CHJ04034 | 0.86 ± 0.45 | 3.06 ± 1.10 | 0.65 ± 0.14 | 1.16 ± 0.94 |
| CHJ04036 | 17.84 ± 3.62 | 15.5 ± 2.56 | 18.36 ± 1.10 | 3.67 ± 0.41 |
| CHJ04058 | 245.31 ± 2.40 | 101.71 ± 1.48 | 56.21 ± 0.55 | 53.96 ± 2.14 |
| CHJ04059 | 10.63 ± 3.37 | 3.26 ± 1.21 | 3.23 ± 0.49 | 3.92 ± 0.56 |
| CHJ04060 | — | — | — | — |
| CHJ04061 | 333.10 ± 2.7 | 143.47 ± 3.1 | 62.13 ± 4,21 | 97.14 ± 4.21 |
| CHJ04064 | 21.50 ± 3.65 | 17.05 ± 0.24 | 9.24 ± 2.65 | 3.55 ± 2.14 |
| CHJ04065 | 25.48 ± 2.11 | 27.07 ± 1.62 | 10.82 ± 1,79 | 3.14 ± 1.06 |
| CHJ04066 | 29.90 ± 2.45 | 77.07 ± 4.07 | 10.29 ± 1.40 | 2.50 ± 0.65 |
| CHJ04068 | 5.46 ± 1.76 | 4.21 ± 1.23 | 1.54 ± 1.34 | 0.52 ± 0.40 |
| CHJ04072 | 12.53 ± 2.44 | 9.62 ± 4.23 | 2.26 ± 0.37 | 5.21 ± 2.31 |
| CHJ04082 | 22.26 ± 1.98 | 28.54 ± 5.22 | 7.51 ± 2.62 | 2.82 ± 1.05 |
| CHJ04083 | 13.35 ± 2.02 | 10.36 ± 1.43 | 2.44 ± 1.03 | 1.23 ± 0.17 |
| CHJ04084 | 42.69 ± 3.06 | 96.38 ± 2.72 | 16.70 ± 2.48 | 7.54 ± 1.02 |
| CHJ04085 | 28.83 ± 5.12 | 49.22 ± 1.97 | 7.32 ± 1.80 | 1.94 ± 0.79 |
| CHJ04086 | — | — | 34.7 ± 3.42 | — |
| CHJ04089 | 27.78 ± 1.73 | 42.71 ± 2.61 | 15.03 ± 2.37 | 49.50 ± 3.54 |
| CHJ04090 | 35.01 ± 2.21 | 36.4 ± 2.17 | 11.70 ± 2.12 | 34.05 ± 2.59 |
| CHJ04091 | 11.05 ± 1.33 | 17.8 ± 3.11 | 4.01 ± 3.11 | 10.63 ± 3.20 |
| CHJ04092 | 30.16 ± 2.76 | 34.5 ± 2.70 | 11.23 ± 1.05 | 47.91 ± 3.43 |
| CHJ04093 | 20.12 ± 2.01 | 10.24 ± 1.69 | 2.60 ± 1.22 | 14.45 ± 2.14 |
| CHJ04094 | 18.40 ± 3.17 | 15.86 ± 2.11 | 2.05 ± 1.01 | 28.51 ± 4.42 |
| CHJ04097 | 24.38 ± 2.78 | 33.59 ± 2.47 | 14.85 ± 2.77 | 32.10 ± 3.39 |
| CHJ04098 | 35.92 ± 2.09 | 47.40 ± 3.20 | 19.48 ± 2.15 | 29.80 ± 3.72 |
| CHJ04099 | 23.10 ± 2.58 | 37.39 ± 2.48 | 16.16 ± 2.11 | 38.24 ± 2.94 |
| CHJ05001 | 30.69 ± 3.12 | 40.12 ± 3.02 | 15.78 ± 1.73 | 49.10 ± 2.71 |
| CHJ05002 | 35.66 ± 2.71 | 42.96 ± 2.37 | 13.66 ± 2.06 | 33.10 ± 3.27 |
| CHJ05003 | 33.97 ± 2.11 | 35.11 ± 1.35 | 24.92 ± 1.93 | 52.49 ± 1.22 |
| cisplatin | 4.21 ± 1.31 | 1.91 ± 0.43 | 5.91 ± 1.64 | 2.64 ± 1.12 |

### 3. Experiment Result

It is found by observing the above experiment data that the majority of the target compounds perform well in inhibitinghuman lung cancer (A549), human ovarian cancer (SKOV3), human melanoma (A375) and human colon cancer (LOVO) cells. Moreover, it can be seen from the data that the compounds with tetramethyl piperidine on the right are usually more active than those with tetrahydropyrrole, diethylin or pyrrole on the right in fighting off cancer. When the length of chain on the hydrophobic side increases on the left of the compound (CHJ03011 and CHJ03012), the compound is still effective against cancer. Nonetheless, the anti-cancer activity of compound can be lowered prominently by introducing large polar compound to the aromatic ring on the left (CHJ04068 and CHJ04061). It is worth noting that the compound with 3 Br atoms as substitute on the aromatic ring on the left (CHJ04022 and CHJ04068) are the most active, as active as the cis-platinum in the control group.

What is claimed is:

1. A compound of general formula (I), or lts pharmaceutically acceptable salt or its optical isomer, 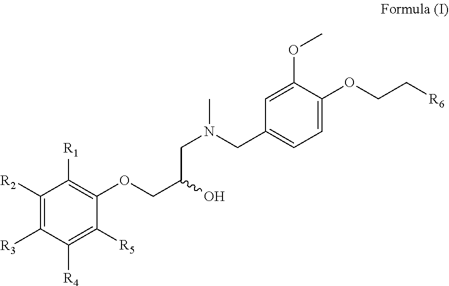
Wherein, R₁ represents —OC₂H₅, —H, —CH(CH₃)₂, —Br, —CF₃—OCH₃,—F, —Cl, —CH₃
R₂ represents —F, —CF₃, —Br, —NHCOCH₃, —Cl, —H, —OCH₃, —CH(CH₃)₂
R₃ represents —H, —CH₃, —Cl, —Br, —NHCOCH₃, —C₃H₇, —F, —C₁₄H₂₉, —OCH₃
R₄ represents —H, —Br, —CF₃, —Cl
R₅ represents —H, —Cl, —I, —Br,
R₆ represents

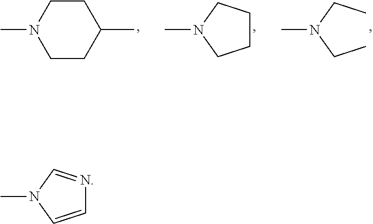

2. The compound described in claim 1 or its pharmaceutically acceptable salt or its optical isomer is characterized in that:
The described R₁ represents —H, —CH(CH₃)₂, —Br, —Cl
R₂ represents —F, —Br, —Cl, —H, —CH(CH₃)₂, —CF₃
R₃ represents —H, —CH₃, —Cl, —Br, —C₁₄H₂₉
R₄ represents —H, —Br, —CF₃, —Cl
R₅ represents —H, —Cl, —I, —Br,
R₆ represents

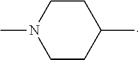

3. The compound described in claim 2 or its pharmaceutically acceptable salt or its optical isomer is characterized in that:
The described R₁ represents —H; R₂ represents —CF₃; R₃ represents —Br; R₄ represents —H; R₅ represents —H;
R₆ represents 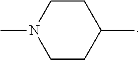
Or
R₁ represents —H; R₂ represents —H; R₃ represents —C₁₄H₂₉; R₄ represents —H; R₅ represents —H;
R₆ represents 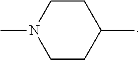
Or
R₁ represents —H; R₂ represents —H; R₃ represents —Cl; R₄ represents —Br; R₅ represents —H;
R₆ represents 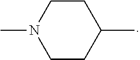
Or
R₁ represents —Br; R₂ represents —H; R₃ represents —Br; R₄ represents —H; R₅ represents —Br;
R₆ represents

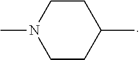

4. The compound described in claim 3 or its pharmaceutically acceptable salt or its optical isomer is characterized. in that:
The described R₁ represents —H; R, represents —CF₃; R₃ represents —Br; R₄ represents —H; R₅ represents —H;
R₆ represents 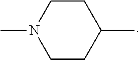
Or
R₁ represents —H; R₂ represents —C₁₄H₂₉; R₄ represents —H; R₅ represents —H;
R₆ represents 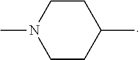
Or
R₁ represents —Br; R₂ represents —H; R₃ represents —Br; R₄ represents —H; R₅ represents —Br;
R₆ represents

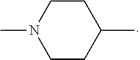

5. the compound described in claim 3 or its pharmaceutically acceptable salt or its optical isomer is characterized in that:
The described R₁ represents —H; represents —CF₃; R₃ represents —Br; R₄ represents —H; R₅ represents —H;
R₆ represents

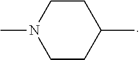

6. The compound described in claim 3 or its pharmaceutically acceptable salt or its optical isomer is characterized in that:
The described R₁ represents —Br; R₂ represents —H; R₃ represents —Br; R₄ represents —H; R₅ represents —Br;
R₆ represents

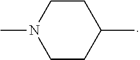

7. Use of the compound of any one of claims 1 or a phamiaceutically acceptable salt thereof, an optical isomer thereof in the preparation of a drug for treating cancer.

8. Based on the application shown it claim 7, it characteristics lie in that the described cancer is lung cancer car ovarian cancer or melanoma or colon cancer.
